(19) United States
(12) Patent Application Publication
(10) Pub. No.: US 2005/0214297 A1
(43) Pub. Date: Sep. 29, 2005

(54) Methods and compositions relating to chitinases and chitinase-like molecules and modulation of osteoclasts

(75) Inventors: Su-Yau Mao, Gaithersburg, MD (US); Wendy White, Germantown, MD (US)

Correspondence Address: JOHNATHAN KLEIN-EVANS, ONE MEDIMMUNE WAY, GAITHERSBURG, MD, 20878, US

(73) Assignee: MEDIMMUNE, INC., Gaithersburg, MD (US)

(21) Appl. No.: 11/063,574

(22) Filed: Feb. 24, 2005

Related U.S. Application Data
(60) Provisional application No. 60/559,089, filed on Apr. 2, 2004.
(60) Provisional application No. 60/547,709, filed on Feb. 25, 2004.

(51) Int. Cl. A61K 39/395

(52) U.S. Cl. 424/145.1

(57) ABSTRACT

The invention relates to the novel discovery that inhibiting a C/CLP modulates (e.g., inhibits) inflammation, as well as, bone and tissue destruction in arthritis. The present invention relates to compositions and methods for the treatment of bone metabolism and connective tissue disorders and diseases (e.g., bone metabolism disorders, osteoarthritis, psoriatic arthritis, rheumatoid arthritis, and osteoporosis) by modulating (e.g., inhibiting) at least one C/CLP. In particular where the inhibition of at least one C/CLP results in the inhibition of osteoclast activity, including but not limited to, osteoclast maturation, osteoclast differentiation, osteoclast proliferation, and osteoclastogenesis. One particular embodiment of the invention relates to inhibiting the expression or activity of one or more C/CLPs.

### CROSS REFERENCE TO RELATED APPLICATIONS

This application claims priority to and benefit of U.S. Provisional Applications No. U.S. 60/547,709 filed Feb. 25, 2004 and 60/559,089 filed Apr. 2, 2004, the disclosures of each of which are incorporated by reference in their entirety for all purposes.

### FIELD OF THE INVENTION

The present invention relates to compounds, compositions and methods for the treatment of bone metabolism and connective tissue disorders or diseases (e.g., Paget's disease, abnormal bone remodeling, osteoporosis, Gorham-Stout syndrome, arthritis (e.g., osteoarthritis, rheumatoid arthritis, psoriatic arthritis), brittle bone disease) by modulating (e.g., inhibiting) osteoclast activity, including but not limited to, osteoclast maturation, osteoclast differentiation, osteoclast proliferation, and osteoclastogenesis.

In one preferred embodiment of the invention, a method of inhibiting osteoclast activity by inhibiting the expression or activity of one or more chitinase/chitinase-like protein (e.g., Ym1 (also known as ECF-L), Ym2, oviductal glycoprotein 1, cartilage glycoprotein 1 (also referred to YKL-40), chitotriosidase, cartilage glycoprotein-39, chondrocyte protein 39 (also known as YKL-39), acidic mammalian chitinase (also referred to as AMCase), TSA1902-L, TSA1902-S) is provided.

In another preferred embodiment of the invention, a method of inhibiting osteoclast activity by both inhibiting the expression or activity of one or more chitinase/chitinase-like protein (e.g., Ym1, AMCase) and inhibiting the expression or activity of one or more other chemokines known to modulate osteoclast activity (e.g., nuclear factor kappa B (RANK) ligand) is provided.

In yet another preferred embodiment, a method of inhibiting osteoclast activity by reducing the activity of chitinase/chitinase-like protein with an antibody (or fragment thereof) alone or in combination with another molecule (e.g., osteoprotegerin (OPG)), which inhibits one or more other chemokines known to modulate osteoclast activity (e.g., RANK ligand).

In another preferred embodiment of the invention, pharmaceutical compositions for the treatment or prevention of inflammatory diseases comprising antibodies that bind one or more chitinase/chitinase-like proteins (e.g., Ym1, AMCase, Chitotriosidase) are provided.

### BACKGROUND OF THE INVENTION

Osteoclasts (OCLs) are multinucleated giant cells that resorb bone and are derived from cells in the monocytic lineage (Kurihara et al., 1990, Endocrinology 126:2733-41). A number of factors that control osteoclastogenesis have been reported including soluble cytokines and membrane bound factors on stromal cells and osteoblasts, e.g., the receptor activator of nuclear factor kappa B (RANK) ligand (Roodman, 2001, J Clin Oncology 19:3562-71). The differentiation of OCLs requires the presence of marrow stromal cells and osteoblasts, and cell-to-cell contact between osteoblast and hematopoietic cells is necessary for inducing differentiation of OCLs (Udagawa et al., 1990, PNAS 87:7260-4). RANK ligand is a critical osteoclastogenic factor that is expressed by osteoblasts and marrow stromal cells in response to several osteotropic factors such as 1,25-dihydroxyvitamin D₃ (see, e.g., Biskobing & Rubin, 1993, Endocrinology 132:862-6), PTH, (see, e.g., Takahashi et al., 1988, Endocrinology 122:1373-82), and interleukin-11 (IL-11) (see, e.g., Elias et al., 1995, Endocrinology 136:489-98. RANK ligand binds to its cognate receptor, RANK, which is found on OCLs and their precursors.

While the genetic events controlling OCL formation from mononuclear precursors have not been fully elucidated, ADAM 8 (a disintegrin and metalloproteinase) was identified as an OCL stimulatory factor, which can increase mouse OCL formation and bone resorption (Choi et al., 2001, J Bone Miner Res 16: 814-22). Further, Oba et al. (2003, J Bone Miner Res.; 18:1332-41) report the identification and characterization of a novel osteoclastogenic cytokine, eosinophil chemotactic factor-L (ECF-L). ECF-L is a member of the chitinase/chitinase-like family of molecules and is highly expressed in OCLs. ECF-L was originally identified as a chemoattractant factor produced by mouse splenocytes that enhances chemotaxis of eosinophils, and attracts not only eosinophils but also T-lymphocytes and bone marrow cells (Owhashi et al., 2000, J Biol Chem 275:1279-86). ECF-L is expressed in spleen, bone marrow, lung, and heart. ECF-L is also recognized in the literature and referred to herein as Ym1. Ym1 has been identified as a macrophage protein, and is transiently synthesized and secreted by activated macrophages during development (Chang et al., 2001 J Biol Chem 276:17497-506).

Ym1 as well as other members of the chitinase-like family have been implicated in the pathogenesis of respiratory diseases (see, e.g. Webb et al., 2004, J Immunol. 172: 1092-8; Sandler et al., 2003, J Immunol. 171: 3655-67; Webb et al., 2001, J Biol. Chem. 276: 41969-76; Boot et al., 2001, J Biol. Chem. 276: 6770-8; Guo et al., 2000, J Biol. Chem. 275:8032-7 and U.S. Patent Publication 2003/0049261 herein incorporated by reference in its entirety). Chitinase gene expression has also been linked to many other types of inflammatory diseases. For instance, rheumatoid arthritis patients demonstrating a remission of active disease showed a significant decrease in serum YKL-40, while patients who changed from inactive to active disease showed an increase in serum YKL-40 (Johansen et al., 1999, Rheumatology 38: 618-26). In other studies, serum and synovial fluid levels of YKL 40 were correlated to the severity of the disease for rheumatoid arthritis, osteoathritis and ankylosing spondylitis (Morgante et al., 1999, Minerva Med. 90:437-41; Morgante et al., 2001, Minerva Med. 92:151-3; D'Amore et al., 2000, Minerva Med. 91:59-68). Correlation has also been seen in tissue remodeling disorders (see, e.g., Johansen et al., 1993, British J of Rheum 32:949-55, Hakala et al., 1993, J Biol Chem 268:25803-10). One group has suggested a physiological role for YKL-40 in limiting the catabolic effects of inflammatory cytokines in patients with inflammatory arthritis (Ling and Recklies, 2004, Biochem. J. 380:651-9). Furthermore, prominent expression of Ym1 was observed in early myeloid precursor cells of hematopoietic tissues such as fetal liver, spleen and bone marrow (Hung et al., 2002, J leuko Biol 72:72-82) as well as by activated macrophages (Chang et al. supra). Thus, it is likely that chitinase/chitinase-like proteins play important roles in many chronic inflammatory diseases especially those that involve tissue damage and remodeling including bone metabolism and connective tissue disorders and diseases.

Rheumatoid arthritis (RA) is one such disease with a high prevalence, affecting 0.5-1% of the population worldwide. Currently anti-TNF-α therapy accounts for 98% of the biologic treatment, but 30% of the patients fail to respond to this therapy. Therefore, there is a great need for treatments with alternate mechanisms of action. It has been demonstrated that OCLs are essential for TNF-α-mediated joint destruction (Redlich et al., 2002, J Clin Invest. 110: 1419-27). Thus, preventing OCL maturation by inhibition the chitinase/chitinase-like family of molecules is one alternative therapy that is addressed by the present invention.

Roodman et al., in International Publication WO 2004/020606 disclose the identification of Ym1 (ECF-L) as an osteoclast stimulating co-factor acting at the later stages of OCL formation. They purport that an anti-Ym1 antibody or Ym1-antisense S-oligonucleotide inhibits OCL formation in marrow cultures. However, no in vivo data was provided.

We demonstrate here, for the first time, that blocking a chitinase/chitinase-like protein, in vivo results in protection of bone and cartilage as well as a reduction in weight loss in a mouse RA model. These results support the role of chitinase/chitinase-like proteins in chronic inflammatory diseases and more specifically the role of chitinase/chitinase-like proteins in OCL-related diseases including bone metabolism and connective tissue disorders and diseases. Furthermore, these results validate human chitinase/chitinase-like proteins as potential therapeutic targets for the prevention and treatment of OCL-related diseases.

It is also known that bone is a frequent site of cancer metastasis, which can result in bone destruction or no bone formation. Bone destruction, mediated by factors produced or induced by tumor cells, stimulated by formation and activation of osteoclasts, which are the normal bone-resorbing cells (Roodman, 2001, J Clin Oncol. 19:3562-71) suggesting that prevention of OCL formation or inhibition of OCL activation and/or activity by inhibition of one or more chitinase/chitinase-like proteins may be an effective therapy to treat cancer metastasis of the bone.

The foregoing teachings satisfy a real and substantial need for a method of minimizing the negative effects of osteoclast formation by inhibiting the formation, activation and/or activity of mature osteoclast cells.

### SUMMARY OF THE INVENTION

The present invention provides novel compositions and methods of inhibiting the formation, activation and/or activity of osteoclasts (e.g., osteoclast maturation, osteoclast differentiation, osteoclast proliferation, osteoclastogenesis) by inhibiting the activity or expression of one or more of the chitinase/chitinase-like protein family members, referred to herein as “C/CLP(s).” C/CLPs include, but are not limited to, eosinophil chemotactic factor-L (ECF-L), Ym1, Ym2, acidic mammalian chitinase (AMCase), oviductal glycoprotein 1, cartilage glycoprotein 1, chitotriosidase, mucin 9, cartilage 10 glycoprotein-39, chondrocyte protein 39, TSA1902-L, and TSA1902-S.

The present invention further includes a method of preventing or treating bone metabolism and connective tissue disorders or diseases or disorders (e.g., Paget's disease, abnormal bone remodeling, osteoporosis, Gorham-Stout syndrome, arthritis (e.g., osteoarthritis, rheumatoid arthritis, psoriatic arthritis), brittle bone disease) in a mammal wherein the disease is associated with an increased level of a chitinase/chitinase-like protein (C/CLP).

The present invention further includes a method of preventing or treating bone metabolism and connective tissue disorders or diseases or disorders (e.g., Paget's disease, abnormal bone remodeling, osteoporosis, Gorham-Stout syndrome, arthritis (e.g., osteoarthritis, rheumatoid arthritis, psoriatic arthritis), brittle bone disease) in a mammal wherein the activity of osteoclasts causes or exacerbates (e.g., causes and/or facilitates bone and/or cartilage destruction or inflammation) said connective tissue disease.

In one preferred embodiment, the method of preventing or treating bone metabolism and connective tissue disorders and diseases comprises administering an effective amount of a C/CLP inhibitor to the mammal alone or in combination with other chemokine modulators (e.g., osteoprotegrin, RANK ligand inhibitors, HMGB1 antagonists, anti-TNF-α), thereby preventing and/or treating the bone metabolism or connective tissue disease or disorder.

In another preferred embodiment, the method of preventing or treating bone metabolism and connective tissue diseases comprises administering an effective amount of a C/CLP inhibitor to the mammal alone or in combination with other molecules used to treat bone metabolism disorders (e.g., calcium supplements, phosphate, aluminum hydroxide, aluminum carbonate gels, magnesium, vitamin D, active forms of Vitamin D (e.g., calcitriol (1,25 dihydroxycholecalciferol), vitamin D₂ (ergocalciferol), vitamin D₃ (cholecalciferol), calcium, lithium, glucocorticoids, plicamnycin (mithramycin), gallium nitrate, hormones (e.g., estrogen, progestin and calcitonin), estrogen antagonists (e.g., tamoxifen), estrogen receptor modulators, androgen receptor modulators, cytotoxic or antiproliferative agents, matrix metalloproteinase inhibitors, inhibitors of epidermal-derived, fibroblast-derived, or platelet-derived growth factors, inhibitors of vascular endothelial growth factor (VEGF), antibodies to a growth factor or to a growth factor receptor, inhibitors of Flk-1/KDR, Flt-1 (VEGF receptors), inhibitors of Tck/Tie-2, or Tie-1 (tyrosine protein kinase receptors), cathepsin K inhibitors, inhibitors of osteoclast proton ATPase, inhibitors of urokinase plasminogen activator (u-PA), tumor-specific antibody-interleukin-2 fusion proteins, prenylation inhibitors, farnesyl transferase inhibitors, geranylgeranyl transferase inhibitors or dual farnesyl/geranylgeranyl transferase inhibitors, parathyroid hormone or parathyroid hormone fragments (a non-limiting example is exogenous PTH analogue, 1-34 PTH), growth hormones, molecules disclosed in U.S. Pat. Nos. 6,472,402 and 6,482,411, renal dialysis, raloxifene HCl (e.g., Evista®V), bisphosphonates (e.g., Actonel®, Aredia®, Didronel®, Fosamax® and Skelid®), thereby preventing and/or treating the connective tissue disease or disorder.

Another preferred embodiment of the invention includes a method of preventing bone cancer metastasis in a mammal, wherein the activity of a C/CLP is inhibited.

Yet another preferred embodiment of the invention includes a method of protecting osteoclast precursors from the effects of chemotherapy, wherein the activity of a C/CLP on osteoclast or osteoclast precursors is inhibited.

Another embodiment of the invention includes a method of inhibiting osteoclast activity by reducing the activity of C/CLPs with an specific inhibitor of a C/CLP such as an antibody or antibody fragment alone or in combination with a molecule (e.g., osteoprotegerin (OPG)), which inhibits one or more other chemokines known to modulate osteoclast activity (e.g., RANK ligand).

Another embodiment of the invention includes a method of inhibiting osteoclast activity by reducing the activity of C/CLPs with a specific inhibitor and/or antagonist of a C/CLP such as a monoclonal antibody, antisense oligonucleotides, RNAi, aptamers, small molecules, small peptides, OPG RANK-Fc and any combinations thereof, as well as other inhibiting materials. Small peptide antagonists of the invention can be identified by screening commercially available peptide libraries by methods that are well known in the art (see for example Koolpe et al., 2002, J Biol Chem 277:46974-9).

Another embodiment of the invention includes a method of inhibiting bone and/or cartilage damage by reducing the activity of C/CLPs with a specific inhibitor of a C/CLP such as a monoclonal antibody, antisense oligonucleotides, RNAi, aptamers, small molecules, OPG RANK-Fc and any combinations thereof, as well as other inhibiting materials.

In another embodiment, osteoclast formation is inhibited by inhibiting RANKL formation. In a further embodiment, a method of inhibiting osteoclast formation is accomplished by means of inhibiting C/CLP activity in the presence of RANKL. In a preferred embodiment, the inhibitor of a C/CLP is an anti-C/CLP antibody. In another preferred embodiment, the anti-C/CLP antibody or active fragment thereof, is a monoclonal antibody, including but not limited to, chimeric, human and humanized antibodies. In further preferred embodiments, such antibodies and fragments inhibit or neutralize C/CLP activity and thereby inhibit osteoclast formation, activation and/or activity. Such antibody fragments include, but are not limited to, scFV, Fab and F(ab′)2 fragments.

One preferred embodiment of the invention is a composition for the treatment or prevention of the inflammation associated with bone metabolism and connective tissue disorders or diseases or disorders (e.g., Paget's disease, abnormal bone remodeling, osteoporosis, Gorham-Stout syndrome, arthritis (e.g., osteoarthritis, rheumatoid arthritis, psoriatic arthritis), brittle bone disease) comprising antibodies that bind C/CLP (e.g., Ym1, AMCase, Chitotriosidase). One preferred embodiment of the invention is a composition for the treatment or prevention of the inflammation associated with bone metabolism and connective tissue disorders or diseases or disorders (e.g., Paget's disease, abnormal bone remodeling, osteoporosis, Gorham-Stout syndrome, arthritis (e.g., osteoarthritis, rheumatoid arthritis, psoriatic arthritis), brittle bone disease) comprising antibodies that bind C/CLP (e.g., Ym1, AMCase, Chitotriosidase) and another therapeutic (e.g., immunomodulatory agents). In another preferred embodiment the composition inhibits inflammation by at least 4 fold, or at least 3 fold, or at least 2 fold, or at least 1 fold. In still another preferred embodiment the composition inhibits and/or prevents connective tissue and bone damage by at least 4 fold, or at least 3 fold, or at least 2 fold, or at least 1 fold.

One preferred embodiment of the invention is an antibody which specifically binds both the mouse ortholog of a C/CLP and a human C/CLP. Another preferred embodiment of the invention is an antibody which specifically binds a human C/CLP versus mouse orthologs. Another preferred embodiment of the invention is an antibody which specifically binds a human C/CLP, but does not substantially bind the corresponding mouse orthogs.

One preferred embodiment of the invention is an antibody which binds both the mouse Ym1 and human Ym1. Another preferred embodiment of the invention is an antibody that binds a C/CLP at least 70%, or at least 80%, or at least 90%, or at least 92%, or at least 95% identical to mouse or human Ym1, wherein said antibody prevents and/or inhibits inflammation and/or damage to cartilage and bone when administered to an individual in need of such therapy. One preferred embodiment of the invention is an antibody which specifically binds both the mouse Ym1 and human Ym1. Another preferred embodiment of the invention is an antibody that binds a C/CLP at least 70%, or at least 80%, or at least 90%, or at least 92%, or at least 95% identical to mouse or human Ym1, wherein said antibody modulates (e.g., inhibits) osteoclast activity (e.g., osteoclastogenesis, proliferation or differentiation). Another preferred embodiment of the invention is an antibody which specifically binds human Ym1 versus mouse Ym1. Another preferred embodiment of the invention is an antibody which specifically binds human Ym1 and does not substantially bind mouse Ym1. Still another preferred embodiment of the invention is an antibody which binds both Ym1 and Ym2. Yet another preferred embodiment of the invention is an antibody which binds fragments of a C/CLP (e.g., catalytic domain, chitin binding domain).

Inhibitors of C/CLP of the invention include, without limitation, antibodies, peptides, aptamers, small molecules, antisense molecules, inhibitory RNA, and ribozymes, siRNA, which are capable of inhibiting or preventing 1) the activity of a C/CLP on osteoclast activity or development; or 2) the binding of a C/CLP with other binding partners; or 3) the biological activity of a C/CLP; or 4) the expression of a C/CLP by a cell, or 5) inflammation and/or connective tissue damage.

It is another object of the present invention to provide a method for resisting formation of osteoclast cells. It is another object of the present invention to provide such a method, which inhibits the function of mature osteoclast cells. It is a further object of the present invention to provide such a method which inhibits the normal functioning of chitinase-like molecules of the invention and/or the influence of RANK ligand.

It will be appreciated that the present invention provides an effective means of therapeutic use in vivo in humans exhibiting OCL formation based upon C/CLP (e.g., Ym1, AMCase, YKL-40) or RANKL.

It is yet another object of the present invention to provide compositions and methods for the prevention and treatment of bone metabolism and connective tissue disorders or diseases or disorders (e.g., Paget's disease, abnormal bone remodeling, osteoporosis, Gorham-Stout syndrome, arthritis (e.g., osteoarthritis, rheumatoid arthritis, psoriatic arthritis), brittle bone disease), said compositions comprising a C/CLP inhibitor. It is a further object of the present invention to provide compositions and methods for the prevention and treatment of inflammation-mediated tissue damage resulting from a bone metabolism or connective tissue disorder or disease, e.g., arthritis.

The present invention also encompasses compositions comprising a C/CLP inhibitor of the in combination with one or more inhibitors of osteoclast activity (e.g., osteoprotegrin) or optionally another therapy (e.g., another prophylactic or therapeutic agent) that is not an C/CLP inhibitor.

The present invention encompasses treatment protocols for bone metabolism and connective tissue diseases or disorders wherein a therapeutic dose of a composition of the invention is administered to a subject in need of such treatment.

These and other objects of the invention will be more fully understood from the following description of the invention with reference to the drawings appended hereto.

### BRIEF DESCRIPTION OF THE DRAWINGS

For the purpose if illustrating the invention, there are depicted in the drawings certain embodiments of the invention. However, the invention is not limited to the precise arrangements and instrumentalities of the embodiments depicted in the drawings.

FIG. 1. Amino Acid Analysis of the C/CLP family members. Panel 1A is an alignment of Ym1 with other members of the mouse C/CLP family. Ym1, Ym2, mouse AMCase and mouse chitotriosidase are compared starting with the first residue of the initial translation product. Identical amino acids are shaded. The double dashed underlined residues indicate the sequence regions that correspond to the putative active site in bacterial chitinases, the amino acid marked with an asterisk is the required catalytic E residues of the chitinase DXDXE consensus. Panel 1B is an alignment of Ym1 with other members of the human C/CLP family. Ym1, humanYKL-39, human YKL-40 human AMCase and human chitotriosidase are compared starting with the first residue of the of the initial translation product. Identical amino acids are shaded. The double underlined residues indicate the sequence regions that correspond to predicted immunogenic domains. The dotted line indicates the chitin-binding domain; the amino acid marked with an asterisk is the required catalytic E residues of the chitinase DXDXE consensus. Panel C represents a series of plots using the a number of algorithms to examine various predicted structures and features of the Ym1 protein. The calculated values for each amino acid are found in FIG. 6.

FIG. 2. Anti-Ym1 staining. Panel A shows that Ym1 staining is not detectable above background in the joints from a normal mouse. Panel B shows the intense Ym1 staining of the macrophages present in the joints from a collagen-induced arthritis (CIA) mouse. Solid arrows indicate cartilage. Asterisks indicate the synovium. Arrowheads indicate macrophages.

FIG. 3. Antibody Preventative Therapy in the CIA Mouse Model. Panels A and B illustrate, respectively, the significant improvement in the clinical and histological scores and the significant improvement in relative body weight of mice treated preventatively with anti-Ym1 antibodies compared to controls.

FIG. 4. Antibody Therapeutic Treatment in the CIA Mouse Model. Panels 4A and B illustrate, respectively, the significant improvement in the clinical and histological scores as well as the relative body weight index of mice treated therapeutically with anti-Ym1 antibodies compared to controls.

FIG. 5. Anti-Ym1 Antibody Protects Joints in the CIA Mouse Model. Panels A, B and C illustrate, respectively, histological examination of the joints from normal, control treated and Ym1 treated CIA mice. Significant protection of the joints is seen the joints of mice treated with anti-Ym1 antibody compared to the joints of the control mice. Solid arrows indicate cartilage. Open arrows indicated bone. Asterisks indicate the synovium.

FIG. 6. Amino Acid Analysis of Ym1. Panels A-G calculated values for each amino acid residue of Ym1 as represented in the plots of FIG. 1C.

### DETAILED DESCRIPTION OF THE INVENTION

The invention is based, in part, on the recognition that inhibitors of chitinase/chitinase-like proteins (C/CLPs) reduce the activity of osteoclasts (e.g., inhibit osteoclast maturation and/or inhibit osteoclast differentiation and/or inhibit osteoclastogenesis) and/or inhibit damage to connective tissue associated with arthritic diseases.

Accordingly the present invention provides chitinase/chitinase-like protein inhibitors, referred to herein as “C/CLP inhibitors of the invention” or simply as “C/CLP inhibitors.” A C/CLP inhibitor is any compound that detectably limits the level of a C/CLP in a cell or tissue when compared to the level of the C/CLP in an otherwise identical cell or tissue in the absence of the compound. The level of the C/CLP includes, but is not limited to, the level of expression of a nucleic acid encoding the molecule, the level of C/CLP detectable, and/or the level of C/CLP activity (e.g., chitinase activity, protein or chitin binding activity).

The present invention encompasses treatment protocols for bone metabolism and connective tissue diseases or disorders wherein a therapeutic dose of a C/CLP inhibitor of the invention is administered to a subject in need of such treatment.

The present invention further encompasses treatment protocols for bone metabolism and connective tissue diseases or disorders wherein a therapeutic dose of a C/CLP inhibitor of the invention is administered to a human in combination with one or more inhibitors of osteoclast activity (e.g., osteoprotegrin) or optionally another therapy (e.g., another prophylactic or therapeutic agent) that is not an C/CLP inhibitor.

The data disclosed herein demonstrate that increased expression, presence and/or activity of the C/CLP, Ym1, is associated with disease pathology in a collagen-induced arthritis mouse model. Further, the data disclosed herein demonstrate for the first time, that administration of a inhibitor of a C/CLP, e.g., an antibody to Ym1, provides a therapeutic effect and treats the disease. Indeed, the data demonstrate that administration of the C/CLP inhibitor before or during the onset of disease serves to drastically diminish the pathology and development of the disease. Thus, these studies show for the first time, that inhibiting a C/CLP implicated in tissue remodeling (in vivo) can reduce the damage associated with bone metabolism and connective tissue disorders and diseases (e.g., arthritis and other tissue remodeling diseases). Accordingly, the present invention provides a novel method whereby administration of a C/CLP inhibitor in a mammal afflicted with an osteoclast-related, bone metabolism and connective tissue disorders and diseases (e.g., arthritis and other tissue remodeling diseases) treats and/or prevents the disease when the disease is mediated by, or associated with, a C/CLP, even though the C/CLP may or may not have detectable chitinase activity.

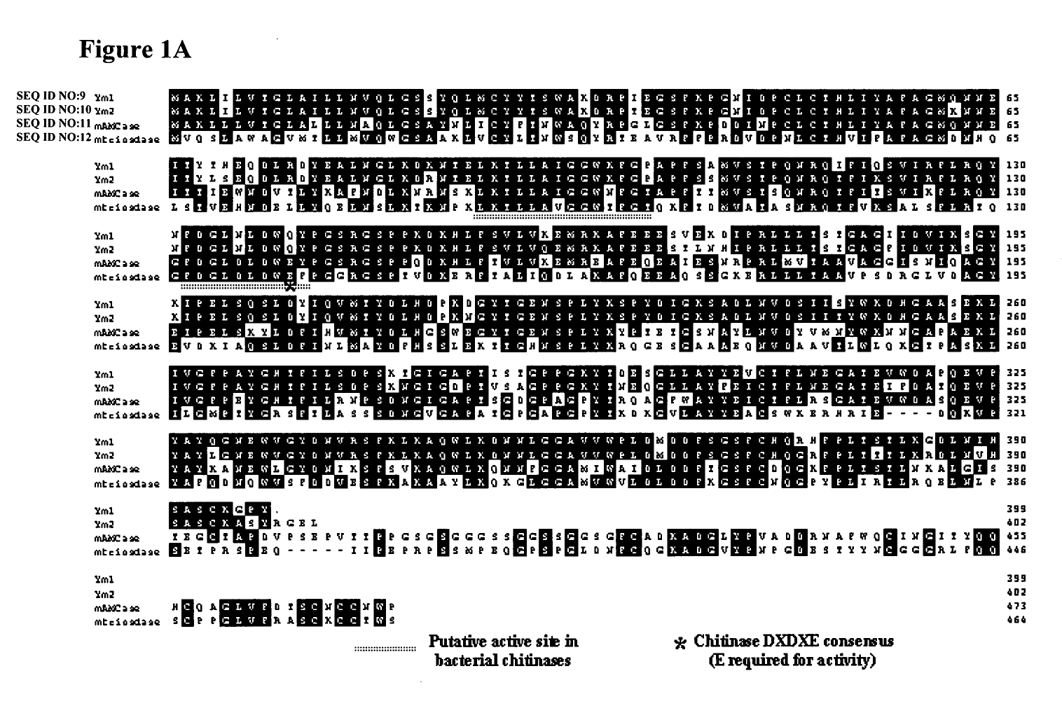

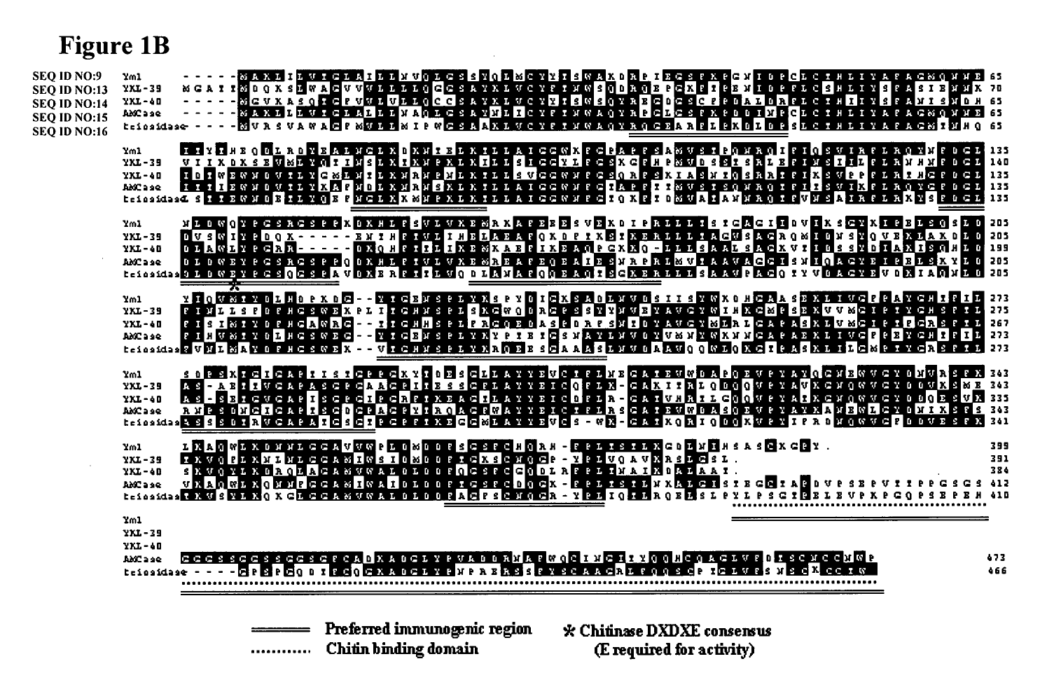

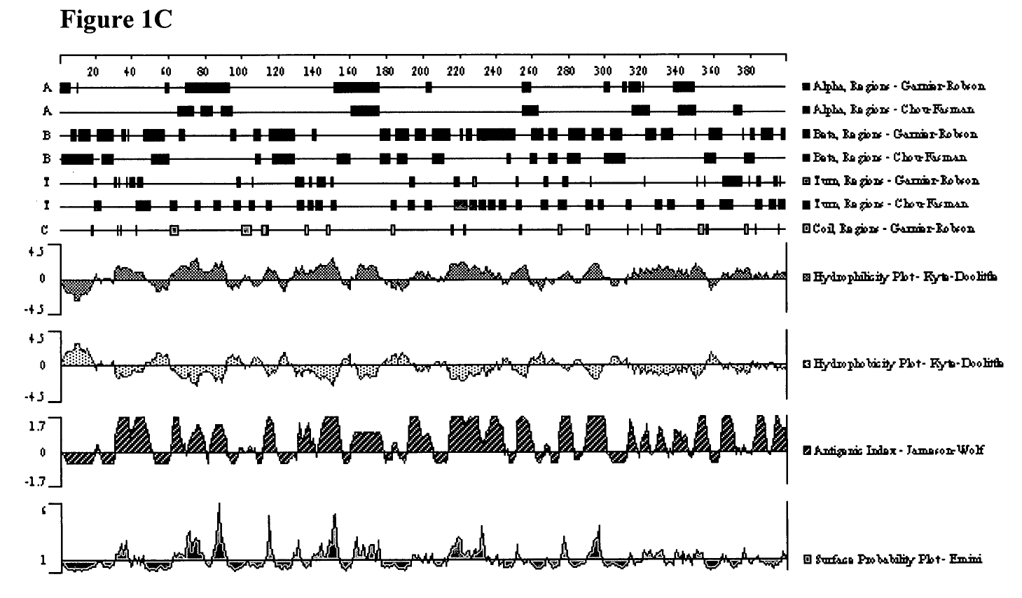

“Chitinase/chitinase-like protein(s)” or “C/CLP(s)”, as the terms are used herein, encompasses a family of polypeptides comprising proteins that are defined by a certain degree of homology to 25 known chitinases, but may or may not demonstrate detectable chitinase activity. In particular, C/CLPs have significant homology to the glycohydrolase family 18 chitinases. A number of such proteins have been identified in mammals including true chitinases which have enzymatic activity such as chitotriosidase, which is expressed in phagocytes, and AMCase (acidic mammalian chitinase), which is abundant in the gastrointestinal tract (Boot et al., 2001, J. Biol. Chem. 276: 6770-8). However, mammals also express genes coding for proteins that show significant amino acid sequence similarity to the true chitinases but which have no enzymatic activity. These proteins have been dubbed “chitinase-like” proteins. These proteins have no chitinase activity due to changes in critical amino acid residues in the putative active center (see FIG. 1), and are thought to have a general function in morphogenesis (Bleau et al., 1999 EXS. 87: 211-21). One of these chitinase-like proteins is oviductin, which is most likely involved in fertilization and protection of the tubal epithelium (Malette et al., 1995 Mol. Reprod. Dev. 41: 384-97). Another is YKL-40 (HCgp39), which is produced in association with tissue remodeling (Volck et al., 1998, Proc. Assoc. Am. Physicians. 110(4): 351-60).

Chitin/chitinase-like proteins (C/CLPs) include, but are not limited to acidic mammalian chitinase (also referred to as eosinophil chemotactic cytokine and exemplified by GenBank Acc. No. AF290003 and No. AF29004), Ym1 (also known as chitinase 3-like 3, ECF-L precursor, as exemplified by GenBank Acc. No. M94584), Ym2 as exemplified by GenBank Acc. No. AF461142, oviductal glycoprotein 1 as exemplified by GenBank Acc. No. XM_{—}131100, cartilage glycoprotein 1 (also referred to as BRP-39, chitinase 3-like 1, GP-39, YKL-40 and exemplified by GenBank Acc. No. X93035), chitotriosidase as exemplified by GenBank Acc. No. NM_{—}003465, oviductal glycoprotein 1 (also referred to as mucin 9, oviductin and as exemplified by GenBank Acc. No. NM_{—}002557), cartilage glycoprotein-39 (also known as chitinase 3-like 1, GP-39, YKL 40, as exemplified by GenBank Acc. No. NM_{—}001276), chondrocyte protein 39 (also known as chitinase 3-like 2, YKL-39, as exemplified by GenBank Acc. No. NM_{—}004000), TSA1902-L as exemplified by GenBank Acc. No. AB025008, TSA1902-S as exemplified by GenBank Acc. No. AB025009 and ECF-L (see, Oba et al., J Bone Miner Res. 2003 7:1332-41). Specifically included in the invention are the full-length C/CLP polypeptides, C/CLP polynucleotides which encode a C/CLP, and fragments thereof. In addition, C/CLP polynucleotides include polynucleotides sharing at least 30% identity, or at least 40% identity, or at least 50% identity, or at least 60% identity, or at least 70% identity, or at least 80% identity, or at least 90% identity, or at least 95% identity, or at least 98% identity, or at least 99% identity to a polynucleotide which encodes a C/CLP (and the complement thereof).

The skilled artisan will further appreciate that a C/CLP is a molecule that exhibits a substantial degree of homology to known chitinases (see, e.g., supra), such that it has been or can be classified as a chitinase family molecule based upon, its amino acid sequence. Further, the skilled artisan would understand, based upon the disclosure provided herein, that while a C/CLP can exhibit homology to a known chitinase, a C/CLP molecule need not demonstrate detectable chitinase activity, in that they may not detectably cleave chitin an in assay known in the art. Such C/CLPs which do not demonstrate detectable chitinase activity, include, but are not limited to, Ym1 (chitinase 3-like 3, ECF-L), Ym2, oviductal glycoprotein 1, cartilage glycoprotein 1 (BRP-39, chitinase 3-like 1, GP-39, YKL-40), oviductal glycoprotein 1 (mucin 9, oviductin), cartilage glycoprotein-39 (chitinase 3-like 1, GP-39, YKL-40), TSA1902-L, TSA1902-Sand chondrocyte protein 39 (chitinase 3-like 2, YKL-39). C/CLPs are not limited to the human polypeptides, but also include the mouse orthologs.

A C/CLP inhibitor can include, but should not be construed as being limited to a chemical compound, a protein, a peptidomemetic, an antibody, a ribozymes, an siRNA, an aptamer and an antisense nucleic acid molecule.

Antibodies or fragments thereof that specifically bind to and inhibit the activity of C/CLPs are specifically preferred embodiments of the invention. Preferably, antibodies or fragments that specifically bind to a C/CLP, or a fragment thereof, do not cross-react with other antigens. Antibodies or fragments that specifically bind to a C/CLP, or a fragment thereof can be identified, for example, by immunoassays or other techniques known to those of skill in the art. Antibodies of the invention include, but are not limited to, synthetic antibodies, monoclonal antibodies, recombinantly produced antibodies, intrabodies, multispecific antibodies, human antibodies, humanized antibodies, chimeric antibodies, synthetic antibodies, single-chain Fvs (scFv), Fab fragments, F(ab′) fragments, disulfide-linked Fvs (sdFv), and anti-idiotypic (anti-Id) antibodies, and derivatives or epitope-binding fragments of any of the above (for review see Chowdhury and Wu, 2005, Methods, in press). In particular, antibodies of the present invention include immunoglobulin molecules and immunologically active portions of immunoglobulin molecules, i.e., molecules that contain an antigen-binding site that specifically binds to a chitinase-like molecule of the invention (e.g., one or more complementarity determining regions (CDRs) of an anti-chitinase-like molecule antibody).

In one embodiment, inhibitory or antagonistic antibodies or fragments that specifically bind to C/CLP, or a fragment thereof, preferentially antagonize the activity of one or more C/CLPs on osteoclasts or osteoclast precursors, and do not significantly antagonize other activities.

“Osteoclast activity”, as used herein, includes but is not limited to, osteoclast maturation, osteoclast differentiation, osteoclast proliferation, osteoclastogenesis, bone resorption, and tissue remodeling.

Polypeptide fragments of the inventions include a peptide or polypeptide comprising an amino acid sequence of at least 5 contiguous amino acid residues, or at least 10 contiguous amino acid residues, or at least 15 contiguous amino acid residues, or at least 20 contiguous amino acid residues, or at least 25 contiguous amino acid residues, or at least 40 contiguous amino acid residues, or at least 50 contiguous amino acid residues, or at least 60 contiguous amino residues, or at least 70 contiguous amino acid residues, or at least contiguous 80 amino acid residues, or at least contiguous 90 amino acid residues, or at least contiguous 100 amino acid residues, or at least contiguous 125 amino acid residues, or at least 150 contiguous amino acid residues, or at least contiguous 175 amino acid residues, or at least contiguous 200 amino acid residues, or at least contiguous 250 amino acid residues of the amino acid sequence of a C/CLP of the invention. Additional embodiments of the present invention are directed to isolated C/CLP polypeptides comprising (or entirely consisting of) amino acids about 35 to 48, or amino acids about 81 to 92, or amino acids about 132 to 149, or amino acids about 161 to 177, or amino acids about 221 to about 240, or amino acids about 274 to 290, or amino acids about 367 to 379, or amino acids 384 to X, where X is any amino acid from 410 to 473 of any one of the chitinase-like molecules represented in FIG. 1B. Optionally, the C/CLP is obtained or is obtainable by expressing the polypeptide encoded by the cDNA insert of the DNA.

Preferred C/CLP Inhibitors of the Invention

The present invention demonstrates that inhibiting a C/CLP is useful for treating or preventing a bone metabolism or connective tissue disorder or disease (e.g., arthritis). Inhibition of a C/CLP prevents, in turn, the pathology associated with a bone metabolism or connective tissue disorder or disease, as amply demonstrated by the data disclosed herein. Surprisingly, a C/CLP inhibitor can be administered to treat the disease even when there is no detectable chitinase activity. Thus, the skilled artisan would appreciate, based upon the disclosure provided herein, that the present invention is not limited to treatment of a disease where detectable chitinase activity is present; instead, the present invention encompasses treatment of a disease associated with or mediated by a C/CLP even when there is no detectable chitinase activity.

In one embodiment, the present invention encompasses inhibition of a C/CLP where the protein does or does not demonstrate detectable chitinase activity in vitro or in vivo. That is, without wishing to be bound by any particular theory, whether the C/CLP demonstrates detectable chitinase activity, either in a cell or tissue or in a cell-free system, is not relevant. More specifically, as demonstrated by the data disclosed herein, a C/CLP, such as Ym1, which does not demonstrate detectable chitinase activity in vitro or in vivo, is expressed at an increased level in disease cells and tissues compared to a cell or tissue that does not demonstrate inflammatory disease pathology, and inhibiting Ym1 using, inter alia, anti-Ym1 antibodies, treats and/or prevents the disease. Such therapeutic effect may be related to inhibition of undetectable chitinase activity, or it may be that the therapeutic effect does not relate to any chitinase or chitinase-like activity of the chitinase-like molecule, and the skilled artisan, based upon the disclosure provided herein, would appreciate that the therapeutic effect can be, but need not be, correlated with any chitinase activity by the molecule.

In one embodiment, the invention relates to inhibiting a C/CLP using various inhibitors. Compounds that inhibit the expression, activity, and/or function of a C/CLP encompass, but are not limited to, an antibody, an antisense nucleic acid, a ribozyme, an siRNA, a small molecule, an aptamer, a peptidomimetic and a chemical compound, either known or to be developed, which inhibits a C/CLP, and thereby a bone metabolism or connective tissue disorder or disease, in particular those associated with inflammation and/or tissue remodeling.

In one embodiment, a C/CLP inhibitor of the invention includes molecules and compounds that prevent or inhibit the expression, activity or function of a C/CLP in a mammal. For instance, a compound that degrades a C/CLP can decrease its function, and can be an inhibitor as contemplated in the present invention. Furthermore, a compound that inhibits, decreases, and/or abolishes expression of a C/CLP such that the C/CLP is not detectable in the cell or tissue is an inhibitor of the invention.

In another embodiment, the invention is directed to aptamers of a C/CLP (e.g., aptamers of Ym1). As is known in the art, aptamers are macromolecules composed of nucleic acid (e.g., RNA, DNA) that bind tightly to a specific molecular target (e.g., a C/CLP as described herein). A particular aptamer may be described by a linear nucleotide sequence and an aptamer is typically about 15-60 nucleotides in length. The chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers have very high affinities for their targets (e.g., affinities in the picomolar to low nanomolar range for proteins). Aptamers are chemically stable and can be boiled or frozen without loss of activity. Because they are synthetic molecules, they are amenable to a variety of modifications, which can optimize their function for particular applications. For in vivo applications, aptamers can be modified to dramatically reduce their sensitivity to degradation by enzymes in the blood. In addition, modification of aptamers can also be used to alter their biodistribution or plasma residence time.

Selection of aptamers that can bind a C/CLP or a fragment there of (e.g., Ym1 or a fragment thereof) can be achieved through methods known in the art. For example, aptamers can be selected using the SELEX (Systematic Evolution of Ligands by Exponential Enrichment) method (Tuerk and Gold, 1990, Science 249:505-10). In the SELEX method, a large library of nucleic acid molecules (e.g., 10¹⁵ different molecules) is produced and/or screened with the target molecule (e.g., binding specificity for a vertebrate C/CLP polypeptide, fragments of vertebrate C/CLP polypeptides (e.g., chitinase domain), epitopic regions of vertebrate C/CLP polypeptides (e.g., epitopic regions of a C/CLP that are bound by the antibodies of the invention)). The target molecule is allowed to incubate with the library of nucleotide sequences for a period of time. Several methods can then be used to physically isolate the aptamer target molecules from the unbound molecules in the mixture and the unbound molecules can be discarded. The aptamers with the highest affinity for the target molecule can then be purified away from the target molecule and amplified enzymatically to produce a new library of molecules that is substantially enriched for aptamers that can bind the target molecule. The enriched library can then be used to initiate a new cycle of selection, partitioning, and amplification. After 5-15 cycles of this selection, partitioning and amplification process, the library is reduced to a small number of aptamers that bind tightly to the target molecule. Individual molecules in the mixture can then be isolated, their nucleotide sequences determined, and their properties with respect to binding affinity and specificity measured and compared. Isolated aptamers can then be further refined to eliminate any nucleotides that do not contribute to target binding and/or aptamer structure (i.e., aptamers truncated to their core binding domain). See, for example, Jayasena, 1999, Clin. Chem. 45:1628-50 for review of aptamer technology; the entire teachings of which are incorporated herein by reference).

In particular embodiments, the aptamers of the invention have the binding specificity and/or functional activity described herein for the antibodies of the invention. Thus, for example, in certain embodiments, the present invention is drawn to aptamers that have the same or similar binding specificity as described herein for the antibodies of the invention (e.g., binding specificity for a vertebrate C/CLP polypeptide, fragments of vertebrate C/CLP polypeptides (e.g., chitinase domain), epitopic regions of vertebrate C/CLP polypeptides (e.g., epitopic regions of a C/CLP that are bound by the antibodies of the invention)). In particular embodiments, the aptamers of the invention can bind to a C/CLP and inhibit one or more functions and/or activity of the C/CLP.

In yet another embodiment, a C/CLP inhibitor of the invention is a chemical compound. Exemplary compounds include, but are not limited to, allosamidin, glucoallosamidin A, glucoallosamidin B, methyl-N-demthylallosamidin, demthylallosamidin, didemethylallosamidin, styloguanidine, a styloguanidine derivative, dipeptide cyclo-(L-Arg-D-Pro), dipeptide cyclo-(L-Arg-L-Pro), riboflavin, and flavin derivatives. Other contemplated C/CLP inhibitors of the invention are ribozymes, siRNA, antisense molecules, aptamers and antibodies.

Antibody C/CLP Inhibitors

As discussed above, the invention encompasses administration of antibodies (preferably monoclonal antibodies) or fragments thereof that specifically bind to and antagonize/inhibit C/CLP activity. In a preferred embodiment, a C/CLP inhibitor of the invention is an antibody or fragment thereof. Antibody inhibitors of C/CLP are referred to herein as “antibodies of the invention” or “anti-C/CLP antibodies.”

As used herein, the term “specifically binds to a C/CLP” and analogous terms refer to, for example, peptides, polypeptides, proteins, fusion proteins and antibodies or fragments thereof that specifically bind to at least one C/CLP or a fragment thereof. A peptide, polypeptide, protein, or antibody that specifically binds to at least one C/CLP or a fragment thereof may bind to other peptides, polypeptides, or proteins with lower affinity as determined by, e.g., immunoassays, BIAcore, or other assays known in the art. Antibodies or fragments that specifically bind to at least one C/CLP or a fragment thereof may be cross reactive with related antigens. Preferably, antibodies or fragments that specifically bind to at least one C/CLP or a fragment thereof preferentially bind to at least one C/CLP over other antigens, most preferably binding a single member of the C/CLP family of proteins. However, the present invention specifically encompasses antibodies with multiple specificities (e.g., an antibody with specificity for two or more discrete antigens (reviewed in Cao et al., 2003, Adv Drug Deliv Rev 55:171; Hudson et al., 2003, Nat Med 1:129)) in the definition of an antibody that “specifically binds to an C/CLP.” For example, bispecific antibodies contain two different binding specificities fused together. In the simplest case a bispecific antibody would bind to two adjacent epitopes on a single target antigen, such an antibody would not cross-react with other antigens (as described supra). Alternatively, bispecific antibodies can bind to two different antigens, such an antibody specifically binds to two different molecules but not to other unrelated molecules. In addition, an antibody that specifically binds a particular C/CLP (e.g., Ym1) may cross-react with related C/CLPs.

Antibodies or fragments that specifically bind to a C/CLP or a fragment thereof can be identified, for example, by immunoassays, BIAcore, or other techniques known to those of skill in the art. An antibody or fragment thereof binds specifically to a C/CLP or a fragment thereof when it binds to a C/CLP or a fragment thereof with higher affinity than to any cross-reactive antigen as determined using experimental techniques, such as radioimmunoassays (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity.

The present invention encompasses antibodies or fragments thereof that specifically bind to a C/CLP selected from the group consisting of: ECF-L; acidic mammalian chitinase (AMCase); Ym1; Ym2; oviductal glycoprotein 1; cartilage glycoprotein 1; chitotriosidase; oviductal glycoprotein 1; cartilage glycoprotein-39; TSA1902-L, TSA1902-S and chondrocyte protein 39, wherein said antibody or fragment thereof inhibits the activity of the C/CLP. In one embodiment, the antibodies of the invention inhibit the activity of a C/CLP on osteoclast development or activity.

Antibodies of the invention include, but are not limited to, monoclonal antibodies, synthetic antibodies, recombinantly produced antibodies, intrabodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), and epitope-binding fragments of any of the above. In particular, antibodies of the invention include immunoglobulin molecules and immunologically active portions of immunoglobulin molecules, i.e., molecules that contain an antigen binding site that specifically bind to a C/CLP and inhibit C/CLP activity. In one embodiment, antibodies of the invention specifically bind to a C/CLP and inhibit C/CLP activity on osteoclasts (e.g., proliferation, differentiation, osteoclastogenesis). It is contemplated that the antibodies of the invention specifically bind a C/CLP epitope, and/or bind a C/CLP with a Koff of less than 3×10⁻³s⁻¹. The antibodies of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

Antibodies of the invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies of the invention may be monospecific, bispecific, trispecific or of greater multi specificity. Multispecific antibodies may specifically bind to different epitopes of a C/CLP or may specifically bind to both a C/CLP as well as a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-9; U.S. Pat. Nos. 4,474,893, 4,714,681, 4,925,648, 5,573,920, and 5,601,819; and Kostelny et al., 1992, J. Immunol. 148:1547-53.

The antibodies of the invention include derivatives that are modified, i.e., by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention encompasses single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079; which are incorporated herein by reference in their entireties).

The methods of the present invention also encompass the use of antibodies or fragments thereof that have half-lives (e.g., serum half-lives) in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present invention or fragments thereof in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor (see, e.g., International Publication No. WO 97/34631 and U.S. patent application Ser. No. 10/020,354 filed Dec. 12, 2001 entitled “Molecules With Extended Half-Lives, Compositions and Uses Thereof,” which are incorporated herein by reference in their entireties).

Antibodies or fragments thereof with increased in vivo half-lives can be generated by attaching to said antibodies or antibody fragments polymer molecules such as high molecular weight polyethylene glycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

Other antibodies specifically contemplated are “oligoclonal” antibodies. As used herein, the term “oligoclonal” antibodies” refers to a predetermined mixture of distinct monoclonal antibodies. See, e.g., International Publication No. WO 95/20401; U.S. Pat. Nos. 5,789,208 and 6,335,163, each of which are incorporated by reference herein. Preferably oligoclonal antibodies consist of a predetermined mixture of antibodies against one or more epitopes are generated in a single cell. More preferably oligoclonal antibodies comprise a plurality of heavy chains capable of pairing with a common light chain to generate antibodies with multiple specificities (e.g., International Publication No. WO 04/009618 which is incorporated by reference herein). Oligoclonal antibodies are particularly useful when it is desired to target multiple epitopes on a single target molecule (e.g., C/CLP). Those skilled in the art will know or can determine what type of antibody or mixture of antibodies is applicable for an intended purpose and desired need.

The present invention also encompasses antibodies that are bispecific. In a preferred embodiment, antibodies of the invention are bispecific T cell engagers (BiTEs). Bispecific T cell engagers (BiTE) are bispecific antibodies that can redirect T cells for antigen-specific elimination of targets. A BiTE molecule has an antigen-binding domain that binds to a T cell antigen (e.g. CD3) at one end of the molecule and an antigen binding domain that will bind to an antigen on the target cell. A BiTE molecule was recently described in International Publication No. WO 99/54440, which is herein incorporated by reference. This publication describes a novel single-chain multifunctional polypeptide that comprises binding sites for the CD19 and CD3 antigens (CD19×CD3). This molecule was derived from two antibodies, one that binds to CD19 on the B cell and an antibody that binds to CD3 on the T cells. The variable regions of these different antibodies are linked by a polypeptide sequence, thus creating a single molecule. Also described, is the linking of the variable heavy chain (VH) and light chain (VL) of a specific binding domain with a flexible linker to create a single chain, bispecific antibody.

In an embodiment of this invention, an antibody or ligand that specifically binds to a C/CLP will comprise a portion of the BiTE molecule. For example, the VH and/or VL (preferably a scFV) of an antibody that binds a chitinase-like molecule can be fused to an anti-CD3 binding portion such as that of the molecule described above, thus creating a BiTE molecule that targets a C/CLP. In another embodiment, the BiTE molecule can comprise a molecule that binds to other T cell antigens (other than CD3). For example, ligands and/or antibodies that specifically bind to T-cell antigens like CD2, CD4, CD8, CD11a, TCR, and CD28 are contemplated to be part of this invention. This list is not meant to be exhaustive but only to illustrate that other molecules that can specifically bind to a T cell antigen can be used as part of a BiTE molecule. These molecules can include the VH and/or VL portions of the antibody or natural ligands (for example LFA3 whose natural ligand is CD3).

Antibody Conjugates

The present invention encompasses the use of antibodies (or fragments thereof) of the invention recombinantly fused or chemically conjugated (including both covalent and non-covalent conjugations) to a heterologous polypeptide (or portion thereof, preferably to a polypeptide of at least 10, at least 20, at least 30, at least 40, at least 50, at least 60, at least 70, at least 80, at least 90 or at least 100 amino acids) to generate fusion proteins. The fusion does not necessarily need to be direct, but may occur through linker sequences. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., International Publication No. WO 93/21232; EP 439,095; Naramura et al., 1994, Immunol. Lett. 39:91-9; U.S. Pat. No. 5,474,981; Gillies et al., 1992, PNAS 89:1428-32; and Fell et al., 1991, J. Immunol. 146:2446-52, each of which are incorporated by reference in their entireties.

The present invention further includes compositions comprising heterologous polypeptides fused or conjugated to antibody fragments. For example, the heterologous polypeptides may be fused or conjugated to a Fab fragment, Fd fragment, Fv fragment, F(ab)₂ fragment, or portion thereof. Methods for fusing or conjugating polypeptides to antibody portions are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-9; Zheng et al., 1995, J. Immunol. 154:5590-600; and Vil et al., 1992, PNAS 89:11337-41 (said references incorporated by reference in their entireties).

DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination.

Moreover, the antibodies or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, PNAS 86:821, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, antibodies of the present invention or fragments or variants thereof conjugated to a diagnostic or detectable agent. Such antibodies can be useful for monitoring or prognosing the development or progression of a cancer as part of a clinical testing procedure, such as determining the efficacy of a particular therapy. Such diagnosis and detection can accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

The present invention further encompasses anti-C/CLP antibodies or fragments thereof conjugated to a therapeutic agent.

An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, an anti-C/CLP antibody or fragment thereof may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, Onconase (or another cytoxic RNase), pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol. 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Moreover, an anti-C/CLP antibody can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies'84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody can be conjugated to a second antibody to form an antibody heteroconjugate as described by Segal in U.S. Pat. No. 4,676,980, which is incorporated herein by reference in its entirety.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Methods of Producing Antibodies

The antibodies of the invention can be produced by any method known in the art for the synthesis of antibodies, in particular, by chemical synthesis or preferably, by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a C/CLP of the invention (either the full length protein or a domain thereof, e.g., the chitinase domain) and once an immune response is detected, e.g., antibodies specific for the C/CLP are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Additionally, a RIMMS (repetitive immunization, multiple sites) technique can be used to immunize an animal (Kilpatrick et al., 1997, Hybridoma 16:381-9, incorporated herein by reference in its entirety). Hybridoma clones are then assayed by methods known in the art for cells that secrete antibodies capable of binding a C/CLP or fragment thereof. Ascites fluid, which generally contains high levels of antibodies, can be generated by immunizing mice with positive hybridoma clones.

Accordingly, monoclonal antibodies can be generated by culturing a hybridoma cell secreting an antibody of the invention wherein, preferably, the hybridoma is generated by fusing splenocytes isolated from a mouse immunized with a C/CLP or fragment thereof with myeloma cells and then screening the hybridomas resulting from the fusion for hybridoma clones that secrete an antibody able to bind a C/CLP or fragment thereof.

Antibody fragments which recognize specific C/CLP epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to the C/CLP epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177; Kettleborough et al., 1994, Eur J. Immunol. 24:952-958; Persic et al., 1997, Gene 187:9; Burton et al., 1994, Advances in Immunology 57:191-280; International Application No. PCT/GB91/01134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Publication No. WO 92/22324; Mullinax et al., 1992, BioTechniques 12:864; Sawai et al., 1995, AJRI 34:26; and Better et al., 1988, Science 240:1041 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise a promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.), Genpharm (San Jose, Calif.) and Medarex (Princeton, N.J.) can be engaged to provide human antibodies directed against a selected antigen using technology similar to that described above.

Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, and 4,816,397, which are incorporated herein by reference in their entirety. Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28:489-498; Studnicka et al., 1994, Protein Engineering 7:805; and Roguska et al., 1994, PNAS 91:969), and chain shuffling (U.S. Pat. No. 5,565,332). Each of the above references are incorporated by reference herein in their entirety.

Bioassays

Inhibition of a C/CLP can be assessed using a wide variety of methods, including those disclosed herein, as well as methods well known in the art or to be developed in the future. That is, one skilled in the art would appreciate, based upon the disclosure provided herein, that inhibition of C/CLP expression can be readily assessed using methods that assess the level of a nucleic acid encoding a C/CLP (e.g., mRNA) and/or the level of a C/CLP present in a cell or fluid. Moreover, one skilled in the art would understand that inhibition of a C/CLP can be assessed by determining the inhibition of chitinase enzymatic activity in a cell or bodily fluid as exemplified elsewhere herein and/or using methods well known in the art or to be developed in the future.

The ability of C/CLP inhibitors of the invention (e.g., antibodies of the invention) to inhibit C/CLP biological activity on osteoclasts and/or tissue remodeling may be screened and measured using assays well known in the art. See, e.g., U.S. Pat. Nos. 5,985,832, 5,719,058, 6,093,533, 6,589,528, 5,641,747; Hirayama et al., 2002, J Endocrinol., 175:155-63; Goldring S R, 2000, Curr. Opin. Rheumatol. 12:195-9; Tamura et al., 1993, J Bone Miner Res. 8: 953-60; Wei et al., 2001, Endocrinology. 142: 1290-5; Zou et al., 2001, Journal of Cellular Biochemistry 83:70-83; Fujikawa et al., 1996, Endocrinology. 137: 4058-60; Takahashi et al., 1988, Endocrinology. 122: 1373-82; Wooley et al., 1985, J. Immuno. 135: 2443-51; Bradley et al., 1997, J Clin Invest 100: 2227-2234; Udagawa et al., 1990, PNAS. 87: 7260-4; Crippes et al., 1993, Endocrinology 137: 918-24; and Teitelbaum, 2000, Science. 289: 1504-8 (review paper in osteoclasts differentiation and bone resorption). Each of these references are incorporated herein by reference in their entireties.

Preferred Therapeutic Applications

Bone Metabolism Disorders

The skeleton is a metabolically active organ that undergoes continuous remodeling throughout life. In normal bone, two different types of cells found in bone, namely osteoclasts and osteoblasts, work together to maintain a healthy balance. Osteoclasts destroy old bone and osteoblasts deposit new minerals and build new bone. When bone loss in a subject outweighs bone formation, a bone metabolism disorder may be the cause.

Bone metabolism disorders and the closely related connective tissue disorders include but are not limited to osteoporosis (including estrogen deficiency, immobilization, glucocorticoid-induced and senile osteoporosis), osteopenia, osteodystrophy, Paget's disease, myositis ossificans, Bechterew's disease, malignant hypercalcemia, metatstatic bone disease, periodontal disease, cholelithiasis, nephrolithiasis, urolithiasis, urinary calculus, hardening of the arteries (sclerosis), arthritis, rheumatoid arthritis, bone metastasis, bursitis, neuritis, tetany, and metastatic bone cancers. Bone metabolism disorders and the closely related connective tissue disorders are commonly associated with inflammation and inflammatory related disorders.

Arthritis is the nation's leading cause of disability among Americans over age 15 resulting in an estimated $86.2 billion in health costs annually. In 2005 the number of Americans suffering from an arthritic disease was estimated to be at least 43 million. Arthritis actually refers to more than 100 different diseases that affect areas in or around joints including, but not limited to, osteoarthritis, rheumatoid arthritis, ankylosing spondylitis, gout, scleroderma, fibromyalgia, Lyme Disease, Systemic Lupus Erythematosus, bursitis and other soft tissue diseases. Arthritic diseases share many common clinical features including, but not limited to pain, stiffness, joint inflammation, and joint damage including cartilage and bone deterioration.

Current therapies utilized for the treatment, prevention and amelioration of symptoms associated with an arthritic disease include the use of analgesics for pain relief (e.g., acetaminophen, propoxyphene, mepeidine and morphine), non-steroidal anti-inflammatory drugs (NSAIDs) such as COX-2 inhibitors, steroids (e.g, corticosteriods and prednisone), biological response modifiers (BRMs) such as etanercept, infliximab, adaliumumab and anakinra, and disease modifiying antirheumatic drugs (DMARDs) such as methotrexate, injectable gold, penicillamine, azathioprine, chloroquine, hydroxychloroquine, sulfasalazine and oral gold. Currently, DMARDs, particularly methotrexate (often in combinantion with another drug, particularly biologic response modifiers) are the standard for aggressively treating RA and other severe arthritic diseases.

Bone loss, including osteopenia (bone density is between 1 and 2.5 standard deviations below the young adult mean) and osteoporosis (bone density is greater than 2.5 standard deviations below the young adult mean), are major public health problems resulting in substantial morbidity and an estimated $14 billion in health costs annually. The number of American women suffering from osteopenia is estimated to be 15 million. The number of American women suffering from osteoporosis is estimated to be 8 million. For men, the numbers are 3 million and 2 million, respectively.

Current therapies for osteoporosis include peptides (e.g., calcitonin), bisphosphonates (e.g., alendronate), estrogen receptor modulators (e.g., estrogen, progestin, estradiol, droloxifene, raloxifene, and tamoxifene), androgen receptor modulators (Davis, S. 1999, J. Steroid Biochem. Mol. Biol. 69:177-184 “The Therapeutic Use of Androgens in Women” and Hansen, K. and Tho, S., 1998, Seminars in Repro. Endocrin. 16:129-134 “Androgens and Bone Health”), growth hormone secretagogues, cathepsin K inhibitors (U.S. Pat. No. 5,501,969, issued Mar. 3, 1996; and U.S. Pat. No. 5,736,357, issued Apr. 7, 1998), PPARγ activators (Okazaki, R. et al., 1999, Endocrinology 140:5060-5065), and inhibitors of the osteoclast proton ATPase (Farina, C. et al., 1999, DDT 4:163-172).

In 2005, about 2,570 new cases of cancer of the bones and joints will be diagnosed, and about 1,210 deaths from these cancers are expected. Bone is the third most common site of metastatic disease, primary cancers of bones account for less than 0.2% of all cancers. Cancers most likely to metastasize to bone include breast, lung, prostate, thyroid and kidney. Metastases are established when a single tumor cell or a clump of cells gain access to the blood stream where they can reach the bone marrow through blood vessels, extravasate, multiply and neovascularize. Metastatic bone lesions can be described as osteolytic, osteoblastic and mixed. The osteolytic lesions are most common where the destructive processes outstrip the laying down of new bone resulting in bone degeneration. Treatments for bone metastases generally involve surgery, often resulting in the amputation of the limb, other treatments involve the use of chemotherapeutic agents and radiation therapy. In some cases the use of bisphosphonate medications may slow the growth cancers that have already spread to the bones.

Treatments for other bone metabolism disorders include, but are not limited to calcium supplements, phosphate, aluminum hydroxide, aluminum carbonate gels, magnesium, vitamin D, active forms of Vitamin D (e.g., calcitriol (1,25 dihydroxycholecalciferol), vitamin D₂ (ergocalciferol), vitamin D₃ (cholecalciferol), calcium, lithium, glucocorticoids, plicamycin (mithramycin), gallium nitrate, hormones (e.g., estrogen, progestin and calcitonin), estrogen antagonists (e.g., tamoxifen), estrogen receptor modulators, androgen receptor modulators, cytotoxic or antiproliferative agents, matrix metalloproteinase inhibitors, inhibitors of epidermal-derived, fibroblast-derived, or platelet-derived growth factors, inhibitors of vascular endothelial growth factor (VEGF), antibodies to a growth factor or to a growth factor receptor, inhibitors of Flk-1/KDR, Flt-1 (VEGF receptors), inhibitors of Tck/Tie-2, or Tie-1 (tyrosine protein kinase receptors), cathepsin K inhibitors, inhibitors of osteoclast proton ATPase, inhibitors of urokinase plasminogen activator (u-PA), tumor-specific antibody-interleukin-2 fusion proteins, prenylation inhibitors, farnesyl transferase inhibitors, geranylgeranyl transferase inhibitors or dual farnesyl/geranylgeranyl transferase inhibitors, parathyroid hormone or parathyroid hormone fragments (a non-limiting example is exogenous PTH analogue, 1-34 PTH), growth hormones, molecules disclosed in U.S. Pat. Nos. 6,472,402 and 6,482,411, renal dialysis, raloxifene HCl (e.g., Evista®), bisphosphonates (e.g., Actonel®, Aredia®g, Didronel®, Fosamax® and Skelid®) and surgery.

Although several treatment options are available for bone metabolism and connective tissue disorders, there are multiple drawbacks including toxicity and poor long-term patient compliance. Thus, there is a need in the art for new, more effective therapies for the treatment of bone metabolism and connective tissue disorders. Without wishing to be bound by any particular theory he present invention provides compositions and methods for the treatment of these disorders and diseases by inhibiting C/CLP activity. In particular, the C/CLP inhibitors of the invention may function by modulating (e.g., inhibiting) osteoclast activity, including but not limited to, osteoclast maturation, osteoclast differentiation, osteoclast proliferation, and osteoclastogenesis.

Bisphosphonates

Bisphosphonates are a class of drugs, which restrict the action of osteoclasts and result in a reduction in bone resorption. Bisphosphonates are used to ease bone pain, slow the spread and growth of metastatic cancers, strengthen bones in people with cancer that has affected the bone, reduce the risk of bone fracturing, and treat hypercalcemia (high levels of calcium in the blood). Accordingly, bisphosphonates are administered to patient populations with osteoporosis or cancer, or patient populations otherwise at risk for skeletal events (e.g., myeloma patients or secondary bone cancer patients, such as breast cancer patients where the cancer has spread to the bone or prostate cancer) to reduce the breakdown of bone, risk of fracture and discomfort (bone pain). See e.g., Pavlakis, N. and Stockler, M., 2002, “Bisphosphonates for Breast Cancer”, The Cochrane Library Issue 4; Homik, J. et al., 2002, “Bisphosphonates for Steroid Induced Osteoporosis” The Cochrane Library Issue 4; Wong, R. and Wiffen P., 2002, “Bisphosphonates for the Relief of Pain Secondary to Bone Metastases” The Cochrane Library Issue 4; Djulbegovic B., et al., 2002, “Bisphosphonates for Multiple Myeloma” Cochrane Library Issue 4, each of which is hereby incorporated by reference.

Side effects from biosphosphonate use include drop in calcium levels, change in kidney function, increased pain, bloating, gas, heartburn, nausea, headache, high temperature and chills, and dizziness. Bisphosphonates may also affect vision, leading to blurred vision, occular irritation, non-specific conjunctivitis (pain, epiphoria, photophobia), anterior uveitis, anterior scleritis, episcleritis, and periocular, lid and/or orbital edema. See Fraunfelder and Franufelder, 2003, Am. J. Ophthalmol. 135:219-22. A rare but more serious side effect from bisphosphonate use is liver damage. Symptoms of bisphosphonate-induced liver damage include yellow skin or eyes, dark urine, nausea and vomiting, or loss of appetite. Other side effects include muscle aches, tenderness and weakness. The toxicity and side effects of bisphosphonates underscore the need in the art for more effective therapies with fewer side effects for the treatment, prevention, management and amelioration of cancer and bone metabolism disorders, and conditions associated therewith.

HMG-CoA Reductase Inhibitors

HMG-CoA (3-hydroxy-3-methylglutaryl coenzyme A) reductase inhibitors (a.k.a. statins) are a class of drugs generally used to treat a number of diseases including elevated cholesterol levels. Their efficacy in lowering cholesterol levels is attributed to an ability to slow down the body's ability to make cholesterol in the blood. Studies have also shown that exposure to HMG-CoA reductase inhibitors is associated with a decreased risk of bone fractures in individuals age 50 years and older. Meier et al., 2000, JAMA 283:3205-10. Additionally, HMG-CoA reductase inhibitors are indicated for use in patients with coronary artery disease and have been shown to be effective at lowering LDL levels and preventing cardiovascular events in angioplasty patients. See Serruys, P., et al., 2002, JAMA 287: 3215-22.

Common side effects from HMG-CoA reductase inhibitors include liver damage, stomach upset or pain, diarrhea, constipation, headache, dizziness, skin rash, muscle tenderness or soreness, unexplained muscle pain, general malaise, fatigue and weakness, and fever.

DMARDs and BRMs

DMARDs and BRMs represent newer categories of medicines for used for the treatment of bone metabolism and connective tissue diseases and disorders (e.g., athritic diseases). DMARDs are considered remittive because they can slow down the disease process, though seldom lead to a complete remission. DMARDs are slow-acting drugs which, may take 6 to 8 months to evoke a response and are generally chosen as a second line treatment option after aspirin and NSAIDs fail. Although DMARDs appear to decrease some inflammation though they are not categorized as anti-inflammatory drugs and unlike NSAIDs they do not directly relieve pain, nor reduce fever. BRMs are biological molecules that work by neutralizing targeted cytokines and, in contrast to DMARDs, are used for treating pain and inflammation. BRMs are generally fast acting however, continued use is required to maintain benefit and administration of the drug can be invensive (e.g., intravenous infusion). In addition BRM therapy is very expensive, annual costs can range from $17,000 to $25,000 or more. The use of DMARDs and BRMs, must be considered carefully as they can result in a number of side effect, some of which are severe, including nausea, skin rash, liver toxicity, kidney toxicity, weakness and fatigue, and increased susceptibility to infection.

Methods of Treatment

The present invention provides methods for the prevention or treatment of bone metabolism and connective tissue disorders and diseases (e.g., osteoarthritis, psoriatic arthritis and osteoporosis, infra). An important aspect of the invention is the ability of a C/CLP inhibitor of the invention to inhibit the inflammation and/or connective tissue damage associated with a bone metabolism or connective tissue disorder as demonstrated, for example, by the inhibition of inflammation as well as both bone and cartilage damage in the CIA mouse model. Accordingly, the present invention provides methods of preventing treating, managing or ameliorating the inflammatory response associated with a bone metabolism or connective tissue disorder or one or more symptoms thereof, said methods comprising administering to a subject in need thereof one or more C/CLP inhibitors.

In particular, the present invention includes methods of preventing or treating bone metabolism and connective tissue disorders or diseases (e.g., Paget's disease, abnormal bone remodeling, osteoporosis, Gorham-Stout syndrome, arthritis (e.g., osteoarthritis, rheumatoid arthritis, psoriatic arthritis), brittle bone disease) in a mammal wherein the disease is associated with an increased level of a C/CLP or wherein the activity of osteoclasts causes or exacerbates (e.g., causes and/or facilitates bone and/or cartilage destruction or inflammation) said bone metabolism or connective tissue disorders or diseases.

There are various methods of treatment that are envisioned to be particularly applicable to such conditions as postmenopausal osteoporosis, Paget's disease of bone, bone metastases and destructive rheumatoid arthritis. See, diseases and disorders described supra.

Another aspect of the invention is a method of preventing calcium release from bone. This is expected to be particularly useful for subjects at risk in the general population to develop osteoporosis. Prophylactic treatment is considered important in certain populations at risk such as young females with a family history of osteoporosis, particularly those who are thin and small-boned and/or have a history of tobacco addiction. Other groups include elderly men and women who tend to be prone to bone fracture, particularly hip fractures.

C/CLP inhibitors of the invention (e.g., antibodies) may be administered in several ways, either locally or systemically in pharmaceutically acceptable formulations. Amounts appropriate for administration are determined on an individual basis depending on such factors as age and sex of the subject, as well as physical condition and weight. Such determinations are well within the skill of the practitioner in the medical field.

It is contemplated that C/CLP inhibitors that specifically bind to C/CLPs will find use as therapeutic agents by blocking bone resorption. Such C/CLP inhibitors are readily screened once a particular C/CLP target has been identified. In the case of antibodies this may be achieved using, for example, phage display (as described supra). A number of different animal models have been described to study osteoporosis; bone metastasis; bone resorption; RANK ligand and osteoprotegrin in myeloma; bone destruction in arthritis; bone destruction in arthritis; and tooth destruction: Osteoporosis (see, e.g., U.S. Pat. No. 6,482,411, Lazner et al., 1999, Hum Mol Genet. 8: 1839-46; Marie, 2003, Osteoporosis Int. 14 Suppl 3:S9-12.); bone metastasis (see, e.g., Rosol et al., 2003 Cancer, 97: 748-57; Clezardin, 2002, Semin Oncol. 29:33-42; Kohno et al., 2003, Breast Cancer. 10:33-7; Yoneda, 2000, Cancer. 88:2979-88.); bone resorption (see, e.g., Wattel et al., 2003, Biochem Pharmacol. 65:35-42); RANK ligand and osteoprotegrin in myeloma (see, e.g., Sezer et al., 2003, Blood. 101:2094-8); bone destruction in arthritis (see, e.g., Gravallese, 2002, Rheum Dis. 61:1184-6); and tooth destruction (Wise et al., 2000, J Dent Res. 79:1937-42). All of these references and methods described therein are incorporated by reference herein.

Combination Treatments

The invention provides combination therapies for prevention, treatment or amelioration of one or more symptoms associated with bone metabolism and connective tissue disorders or diseases (e.g., Paget's disease, abnormal bone remodeling, osteoporosis, Gorham-Stout syndrome, arthritis (e.g., osteoarthritis, rheumatoid arthritis, psoriatic arthritis), brittle bone disease) in a subject, said combination therapies comprising administering to said subject one or more C/CLP inhibitor of the invention and one or more prophylactic or therapeutic agents other than a C/CLP inhibitor of the invention. In particular, the invention provides combination therapies for prevention, treatment or amelioration of one or more symptoms associated with an bone metabolism or connective tissue disorder or disease in a subject, said combination therapies comprising administering to said subject a C/CLP inhibitor of the invention, preferably an anti-C/CLP antibody of the invention, and at least one other prophylactic or therapeutic agent which has a different mechanism of action than the C/CLP inhibitor of the invention.

In one preferred embodiment, the method of preventing, treating, or amelioration of one or more symptoms associated bone metabolism or connective tissue disorders or diseases comprises administering an effective amount of a C/CLP inhibitor to the mammal alone or in combination with other chemokine modulators (e.g., osteoprotegrin, RANK ligand inhibitors, HMGB1 antagonists, anti-TNF-αe), thereby preventing and/or treating the bone metabolism or connective tissue disease or disorder. It is further contemplated that C/CLP inhibitors may be used in combination with certain anti-inflammatory treatments including, in particular, anti-TNF-α agents and methotrexate.

In a specific embodiment, the method of preventing, treating or ameliorating of one or more symptoms associated with a bone metabolism or connective tissue disorders or diseases comprises administering an effective amount of a C/CLP inhibitor in combination with effective amount of one or more TNF antagonist. Any TNF-α antagonist known to one of skill in the art can be used. Examples of TNF-α antagonists include, but are not limited to, antibodies (e.g., infliximab (REMICADE™; Centacor), D2E7 (Abbott Laboratories/Knoll Pharmaceuticals Co., Mt. Olive, N.J.), CDP571 which is also known as HUMICADE™ and CDP-870 (both of Celltech/Pharmacia, Slough, U.K.), and TN3-19.12 (Williams et al., 1994, Proc. Natl. Acad. Sci. USA 91:2762-6; Thorbecke et al., 1992, Proc. Natl. Acad. Sci. USA 89:7375-9)) soluble TNF-α receptors (e.g., sTNF-R1 (Amgen), etanercept (ENBREL™; Immunex) and its rat homolog RENBREL™, soluble inhibitors of TNF-α derived from TNFrI, TNFrII (Kohno et al., 1990, Proc. Natl. Acad. Sci. USA 87:8331-85), and TNF-α Inh (Seckinger et al, 1990, Proc. Natl. Acad. Sci. USA 87:5188-92)), IL-10, TNFR-IgG (Ashkenazi et al., 1991, Proc. Natl. Acad. Sci. USA 88:10535-9), the murine product TBP-1 (Serono/Yeda), the vaccine CytoTAb (Protherics), antisense molecule104838 (ISIS), the peptide RDP-58 (SangStat), thalidomide (Celgene), CDC-801 (Celgene), DPC-333 (Dupont), VX-745 (Vertex), AGIX-4207 (AtheroGenics), ITF-2357 (Italfarmaco), NPI31021-31 (Nereus), SCIO-469 (Scios), TACE targeter (Immunix/AHP), CLX-120500 (Calyx), Thiazolopyrim (Dynavax), auranofin (Ridaura) (SmithKline Beecham Pharmaceuticals), quinacrine (mepacrine dichlorohydrate), tenidap (Enablex), Melanin (Large Scale Biological), and anti-p38 MAPK agents by Uriach. The present invention also encompasses the use of antibodies that specifically bind to TNF-α as disclosed in the following U.S. patents in the compositions and methods of the invention: U.S. Pat. Nos. 5,136,021; 5,147,638; 5,223,395; 5,231,024; 5,334,380; 5,360,716; 5,426,181; 5,436,154; 5,610,279; 5,644,034; 5,656,272; 5,658,746; 5,698,195; 5,736,138; 5,741,488; 5,808,029; 5,919,452; 5,958,412; 5,959,087; 5,968,741; 5,994,510; 6,036,978; 6,114,517; and 6,171,787; each of which are herein incorporated by reference in their entirety.

In another preferred embodiment, the method of preventing, treating or ameliorating of one or more symptoms associated with a bone metabolism or connective tissue disorders or diseases comprises administering an effective amount of a C/CLP inhibitor to the mammal alone or in combination with one or more immunomodulatory agents. Any immunomodulatory agent known to one of skill in the art may be used in the methods and compositions of the invention. Immunomodulatory agents can affect one or more or all aspects of the immune response. Aspects of the immune response include, but are not limited to, the inflammatory response, the complement cascade, leukocyte and lymphocyte differentiation, proliferation, and/or effector function, monocyte and/or basophil counts, and the cellular communication among the cells of the immune system. Examples of immunomodulatory agents include, but are not limited to, methothrexate, leflunomide, cyclophosphamide, cyclosporine A, and macrolide antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunamide), T cell receptor modulators, and cytokine receptor modulators. Examples of T cell receptor modulators include, but are not limited to, anti-T cell receptor antibodies (e.g., anti-CD4 monoclonal antibodies, anti-CD3 monoclonal antibodies, anti-CD8 monoclonal antibodies, anti-CD40 ligand monoclonal antibodies, anti-CD2 monoclonal antibodies) and CTLA4-immunoglobulin. Examples of cytokine receptor modulators include, but are not limited to, soluble cytokine receptors (e.g., the extracellular domain of a TNF-α receptor or a fragment thereof, the extracellular domain of an IL-10 receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-7, and GM-CSF), anti-cytokine receptor antibodies (e.g., anti-IL-2 receptor antibodies, anti-IL-4 receptor antibodies, anti-IL-6 receptor antibodies, anti-IL-10 receptor antibodies, and anti-IL-12 receptor antibodies), anti-cytokine antibodies (e.g., anti-IFN receptor antibodies, anti-TNF-α antibodies, anti-IL-10 antibodies, anti-IL-6 antibodies, and anti-IL-12 antibodies).

In still another preferred embodiment, the method of preventing, treating or ameliorating of one or more symptoms associated with a bone metabolism or connective tissue disorders or diseases comprises administering an effective amount of a C/CLP inhibitor to the mammal alone or in combination with one or more anti-inflammatory agents. Anti-inflammatory agents have exhibited success in the treatment of many inflammatory disorders and are now a common and standard treatment for such disorders. Any anti-inflammatory agent known to one of skill in the art can be used. Examples of anti-inflammatory agents include, but are not limited to, non-steroidal anti-inflammatory drugs (NSAIDS), steroidal anti-inflammatory drugs, beta-agonists, anticholingeric agents, and methyl xanthines. Examples of NSAIDS include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

In yet another preferred embodiment, the method of preventing, treating or ameliorating of one or more symptoms associated with a bone metabolism or connective tissue disorders or diseases comprises administering an effective amount of a C/CLP inhibitor to the mammal alone or in combination with other molecules used to treat bone metabolism disorders (e.g., calcium supplements, phosphate, aluminum hydroxide, aluminum carbonate gels, magnesium, vitamin D, active forms of Vitamin D (e.g., calcitriol (1,25 dihydroxycholecalciferol), vitamin D₂ (ergocalciferol), vitamin D₃ (cholecalciferol), calcium, lithium, glucocorticoids, plicamycin (mithramycin), gallium nitrate, hormones (e.g., estrogen, progestin and calcitonin), estrogen antagonists (e.g., tamoxifen), estrogen receptor modulators, androgen receptor modulators, cytotoxic or antiproliferative agents, matrix metalloproteinase inhibitors, inhibitors of epidermal-derived, fibroblast-derived, or platelet-derived growth factors, inhibitors of vascular endothelial growth factor (VEGF), antibodies to a growth factor or to a growth factor receptor, inhibitors of Flk-1/KDR, Flt-1 (VEGF receptors), inhibitors of Tck/Tie-2, or Tie-1 (tyrosine protein kinase receptors), cathepsin K inhibitors, inhibitors of osteoclast proton ATPase, inhibitors of urokinase plasminogen activator (u-PA), tumor-specific antibody-interleukin-2 fusion proteins, prenylation inhibitors, farnesyl transferase inhibitors, geranylgeranyl transferase inhibitors or dual farnesyl/geranylgeranyl transferase inhibitors, parathyroid hormone or parathyroid hormone fragments (a non-limiting example is exogenous PTH analogue, 1-34 PTH), growth hormones, molecules disclosed in U.S. Pat. Nos. 6,472,402 and 6,482,411, renal dialysis, raloxifene HCl (e.g., Evista®), bisphosphonates (e.g., Actonel®, Aredia®, Didronel®, Fosamax® and Skelid®), thereby preventing and/or treating the connective tissue disease or disorder.

It is contemplated that the use of a C/CLP inhibitor in combination with other therapeutic agents may enable lower dosages of the prophylactic or therapeutic agents utilized in conjunction with C/CLP inhibitors of the invention for the preventing, treating or ameliorating of one or more symptoms associated with a bone metabolism or connective tissue disorders or diseases and/or less frequent administration of such prophylactic or therapeutic agents to a subject with a bone metabolism or connective tissue disorder to achieve a prophylactic or therapeutic effect. It is further contemplated that the use of a C/CLP inhibitor in combination with other therapeutic agents may reduce or avoid unwanted or adverse side effects associated with the administration of current single agent therapies and/or existing combination therapies for bone metabolism and connective tissue disorders, which in turn improves patient compliance with the treatment protocol.

The prophylactic or therapeutic agents used in combination with a C/CLP inhibitor of the present invention can be administered concomitantly or sequentially to a subject. The prophylactic or therapeutic agents used in combination with a C/CLP inhibitor of the present invention can also be cyclically administered. Cycling therapy involves the administration of a first prophylactic or therapeutic agent for a period of time, followed by the administration of a second prophylactic or therapeutic agent for a period of time and repeating this sequential administration, i.e., the cycle, in order to reduce the development of resistance to one of the agents, to avoid or reduce the side effects of one of the agents, and/or to improve the efficacy of the treatment.

The prophylactic or therapeutic agents used in combination with a C/CLP inhibitor of the present invention can be administered to a subject concurrently. The term “concurrently” is not limited to the administration of prophylactic or therapeutic agents at exactly the same time, but rather it is meant that a C/CLP inhibitor and the other agent are administered to a subject in a sequence and within a time interval such that the C/CLP inhibitor can act together with the other agent to provide an increased benefit than if they were administered otherwise.

For example, each prophylactic or therapeutic agent (e.g., anti-C/CLP antibody, anti-TNF-α antibody, or methotrexate) may be administered at the same time or sequentially in any order at different points in time; however, if not administered at the same time, they should be administered sufficiently close in time so as to provide the desired therapeutic or prophylactic effect. Each prophylactic or therapeutic agent can be administered separately, in any appropriate form and by any suitable route. In various embodiments, the prophylactic or therapeutic agents are administered less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 6 hours apart, no more than 24 hours apart or no more than 48 hours apart. In preferred embodiments, two or more prophylactic or therapeutic agents are administered within the same patient visit.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

Therapeutic and/or Prophylactic Compositions

The present invention provides compositions (referred to herein as “compositions of the invention”) for the prevention, treatment or amelioration of one or more symptoms associated with a bone metabolism or connective tissue disorders or diseases. In a specific embodiment, a composition comprises one or more C/CLP inhibitor of the invention in a pharmaceutically acceptable carrier. In another embodiment, a composition comprises one or more C/CLP inhibitor of the invention and one or more prophylactic or therapeutic agents other than a C/CLP inhibitor in a pharmaceutically acceptable carrier, said prophylactic or therapeutic agents known to be use for the of having been or currently being used in for the preventing, treating or ameliorating of one or more symptoms associated with a bone metabolism or connective tissue disorders or diseases. Preferably, the compositions of the invention are sterile and in suitable form for a particular method of administration to a subject with a connective tissue disorder.

In one embodiment, a composition of the invention comprises a pharmaceutically acceptable carrier, one or more C/CLP inhibitor of the invention, and one or more immunomodulatory agents. In another embodiment, a pharmaceutical composition comprises a pharmaceutically acceptable carrier, at least one anti-C/CLP antibody of the invention and one or more immunomodulatory agents. In another embodiment, a composition comprises a pharmaceutically acceptable carrier, at least one anti-C/CLP antibody of the invention, and methotrexate.

In a specific embodiment, a composition of the invention comprises a pharmaceutically acceptable carrier, at least one C/CLP inhibitor of the invention, and one or more TNF-α antagonists. In another embodiment, a composition comprises a pharmaceutically acceptable carrier, at least one anti-C/CLP antibody of the invention, and one or more TNF-α antagonists. In a preferred embodiment, a composition comprises a pharmaceutically acceptable carrier, at least one anti-C/CLP antibody of the invention, and a soluble TNF-α receptor (e.g., etanercept) or an antibody that specifically binds to TNF-α (e.g., ENBREL™ or REMICADE™).

In a specific embodiment, a composition of the invention comprises a pharmaceutically acceptable carrier, one or more C/CLP inhibitor of the invention, and one or more anti-inflammatory agents. In another embodiment, a composition comprises a pharmaceutically acceptable carrier, at least one anti-C/CLP antibody of the invention, and one or more anti-inflammatory agents. In a preferred embodiment, a composition comprises a pharmaceutically acceptable carrier, at least one anti-C/CLP antibody of the invention, and a steroidal or non-steroidal anti-inflammatory drug.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents.

These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective 130 amount of a prophylactic or therapeutic agent preferably in purified form, together with a suitable amount of carrier so as to provide the form for proper administration to the patient.

The formulation should suit the mode of administration. In a preferred embodiment, the compositions are sterile and in suitable form for administration to a subject, preferably an animal subject, more preferably a mammalian subject, and most preferably a human subject.

In a preferred embodiment, the composition of the invention is a pyrogen-free formulation which is substantially free of endotoxins and/or related pyrogenic substances. Endotoxins include toxins that are confined inside a microorganism and are released when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, it is advantageous to remove even low amounts of endotoxins from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1): 223 (2000)). When therapeutic proteins are administered in amounts of several hundred or thousand milligrams per kilogram body weight, as can be the case with monoclonal antibodies, it is advantageous to remove even trace amounts of endotoxin. Preferably, endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

Therapeutic and/or Prophylactic Administration

The invention also provides methods for the prevention, treatment or amelioration of one or more symptoms associated with a bone metabolism or connective tissue disorders or diseases by administration to a subject of an effective amount of a compound or composition of the invention, preferably comprising an antibody of the invention. In a preferred embodiment, the compound is substantially purified (e.g., substantially free from substances that limit its effect or produce undesired side effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-32), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see, Langer, 1990, Science 249:1527-33; Treat et al., in Liposomes in the Therapy of Infectious Disease and Cancer, Lopez-Berestein and Fidler (eds.), Liss, New York, pp. 353-365 (1989); Lopez-Berestein, ibid. pp. 317-327; see generally ibid.)

In yet another embodiment, the compound or composition of the invention can be delivered in a controlled release system. In one embodiment, a pump may be used to achieve controlled or sustained release (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used to achieve controlled or sustained release of a pharmaceutical composition. (See Medical Applications of Controlled Release, Langer and Wise (eds.), CRC Press, Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105; U.S. Pat. Nos. 5,679,377; 5,916,597; 5,912,015; 5,989,463; and 5,128,326 and International Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), 131poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-8), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the prevention, treatment or amelioration of one or more symptoms associated with a bone metabolism or connective tissue disorders or diseases can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

In a preferred embodiment of the invention, REMICADE™ is supplied as a sterile and lyophilized powder for intravenous infusion to be reconstituted with 10 ml sterile water for injection. Each single-use vial of REMICADE™ contains 100 mg infliximab, 500 mg sucrose, 0.5 mg polysorbate 80, 2.2 mg monobasic sodium phosphate and 6.1 mg dibasic sodium phosphate. According to The Physician's Desk Reference (55^{th} ed., 2001), the total dose of the reconstituted product must be further diluted to 250 ml with 0.9% Sodium Chloride Injection, USP, with the infusion concentration ranging between 0.4 mg/ml and 4 mg/ml. In an embodiment of the invention, a recommended dose of REMICADE™ is 0.1 to 10 mg/kg, more preferably 1 to 7 mg/kg, even more preferably 2 to 6 mg/kg, and most preferably 3 to 5 mg/kg. In a most preferred embodiment, the dose does not exceed 3 mg/kg′. In certain preferred embodiments, REMICADE™ is administrated by intravenous infusion followed with an additional dose at 2 and 6 weeks after the first infusion then every 8 weeks thereafter.

In another preferred embodiment of the invention, ENBREL™ is supplied as a sterile, preservative-free, lyophilized powder for parenteral administration after reconstitution with 1 ml of supplied Sterile Bacteriostatic Water for Injection, USP (containing 0.9% benzyl alcohol). According to The Physician's Desk Reference (55^{th} ed., 2001) Each single-use vial of ENBREL™ contains 25 mg etanercept, 40 mg mannitol, 10 mg sucrose, and 1.2 mg tromethamine. In one embodiment, the recommended dosage of ENBREL™ is 0.01 to 10 mg/kg, preferably 0.1 to 10 mg/kg, more preferably 0.1 to 5 mg/kg, and even more preferably 0.5 to 2 mg/kg. In another embodiment of the invention, the recommended dose of ENBREL™ is 0.01 to 10 mg/kg/week, more preferably 0.1 to 5 mg/kg/week, and even more preferably 0.5 to 2 mg/kg/week. In a most preferred embodiment, the weekly dose is not to exceed 50 mg/week. In preferred embodiments, ENBREL™ is administrated by subcutaneous injection twice a week.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

It is also specifically contemplated that an antibody of the invention can be used as a diagnostic for monitoring bone metabolism and connective tissue disorders and diseases, such as, for example, joint diseases (e.g., RA), diseases related to loss of connective tissue in bone (e.g., osteoporosis and osteoarthritis), as well as, monitoring treatment efficacy. Diagnostic methods utilizing antibodies to detect and/or measure/quantify the presence of a C/CLP in a cell or bodily fluid (e.g. ELISA assays, dot blots, western blots) are well known in the art.

Having generally described the invention, the same will be more readily understood by reference to the following examples, which are provided by way of illustration and are not intended as limiting.

Characterization and Demonstration of Prophylactic or Therapeutic

In a preferred embodiment, the compounds and compositions of the invention are effective to prevent, reduce or alleviate symptoms of bone metabolism and connective tissue disorders and diseases, in particular, arthritic diseases. Efficacy may be measured by any number of clinical outcomes including, but not limited to, reducing and/or counteracting progressive cartilage destruction, joint inflammation (e.g., infiltration of inflammatory cells into the joint), synovial hyperplasia, progressive bone destruction. Efficacy may also be measured by patient health assessment scores (for review see, Bellamy, 1989, Scand J Rheumatol Suppl. 80:3-16) such as the Keitel function test (Soderlin et al., 1998, J Rheumatol. 25: 1895-9), Cochin scale (Duruöz et al., 1996, J Rheumatol 23:1167-72), Ritchie articular index (Ritchie et al., 1968, Q J Med 37:393-406) and the Arthritis Self-Efficacy Scale (Lorig et al., 1989, Arthitis and Rheumatism 32:37-44).

Tests for monitoring the improvement of a disease can include specific tests directed, for example, to the determination of systemic response to inflammation, which include the erythrocyte sedimentation rate (ESR) and acute phase reactants (APR). Observations are made of the swelling, etc. of the afflicted body parts. Improvement in stiffness, and grip (where applicable), and reduction in pain of the patient is also observed. If the patient's condition is stable, he is re-treated at the same dosage weekly and is evaluated weekly. Provided the patient's condition is stable, the treatment may be continued. After six months of treatment, anatomical changes of the skeleton are determined by radiologic imaging, for example by X-radiography. At the end of each period, the patient is again evaluated. Comparison of the pre-treatment and post-treatment radiological assessment, ESR and APR indicates the efficacy of the treatments. According to the efficacy of the treatments and the patient's condition, the dosage may be increased or maintained constant for the duration of treatment

Several aspects of the compounds and compositions of the invention are preferably tested in vitro, in a cell culture system, and in an animal model organism, such as a rodent animal model system, for the desired therapeutic activity prior to use in humans. For example, assays which can be used to determine whether administration of a specific pharmaceutical composition is indicated, include cell culture assays in which a patient tissue sample is grown in culture, and exposed to or otherwise contacted with a pharmaceutical composition, and the effect of such composition upon the tissue sample is observed. The tissue sample can be obtained by biopsy from the patient. This test allows the identification of the therapeutically most effective prophylactic or therapeutic molecule(s) for each individual patient. In various specific embodiments, in vitro assays can be carried out with representative cells of cell types involved in an autoimmune or inflammatory disorder (e.g., T cells), to determine if a pharmaceutical composition of the invention has a desired effect upon such cell types.

Combinations of prophylactic and/or therapeutic agents can be tested in suitable animal model systems prior to use in humans. Such animal model systems include, but are not limited to, rats, mice, chicken, cows, monkeys, pigs, dogs, rabbits, etc. Any animal system well known in the art may be used. In a specific embodiment of the invention combinations of prophylactic and/or therapeutic agents are tested in a mouse model system. Such model systems are widely used and well known to the skilled artisan. Prophylactic and/or therapeutic agents can be administered repeatedly. Several aspects of the procedure may vary. Said aspects include the temporal regime of administering the prophylactic and/or therapeutic agents, and whether such agents are administered separately or as an admixture.

The efficacy of the compositions of invention to prevent, treat or ameliorate one or more symptoms associated with a bone metabolism or connective tissue disorders or diseases (e.g., anti-inflammatory activity) can be determined by using various experimental animal models of inflammatory arthritis known in the art and described in Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993) incorporated herein by reference in its entirety.

Experimental and spontaneous animal models of inflammatory arthritis and autoimmune rheumatic diseases can also be used to assess the anti-inflammatory activity of the combination therapies of invention. The following are some assays provided as examples and not by limitation, The principle animal models for arthritis or inflammatory disease known in the art and widely used include: adjuvant-induced arthritis rat models, collagen-induced arthritis rat and mouse models and antigen-induced arthritis rat, rabbit and hamster models, all described in Crofford L. J. and Wilder R. L., ibid.

The efficacy of the compositions of invention to prevent, treat or ameliorate one or more symptoms associated with a bone metabolism or connective tissue disorders or diseases (e.g., anti-inflammatory activity) can be assessed, for example, using a carrageenan-induced arthritis rat model. Carrageenan-induced arthritis has also been used in rabbit, dog and pig in studies of chronic arthritis or inflammation. Quantitative histomorphometric assessment is used to determine therapeutic efficacy. The methods for using such a carrageenan-induced arthritis model is described in Hansra P. et al., 2000, Inflammation, 24:141-55. Also commonly used are zymosan-induced inflammation animal models as known and described in the art.

The efficacy of the compositions of invention to prevent, treat or ameliorate one or more symptoms associated with a bone metabolism or connective tissue disorders or diseases (e.g., anti-inflammatory activity) can also be assessed by measuring the inhibition of carrageenan-induced paw edema in the rat, using a modification of the method described in Winter C. A. et al., 1962, Proc. Soc. Exp. Biol Med. 111:544-7. This assay has been used as a primary in vivo screen for the anti-inflammatory activity of most NSAIDs, and is considered predictive of human efficacy. The anti-inflammatory activity of the test prophylactic or therapeutic agents is expressed as the percent inhibition of the increase in hind paw weight of the test group relative to the vehicle dosed control group.

In a specific embodiment of the invention where the experimental animal model used is adjuvant-induced arthritis rat model, body weight can be measured relative to a control group to determine the efficacy of the compositions of invention.

In a specific embodiment of the invention where the experimental animal model used is adjuvant-induced arthritis rat model, joint inflammation and/or destruction can be measured relative to a control group to determine the efficacy of the compositions of invention.

### EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### Example 1

Methods of Measuring Osteoclastogenesis, OCL Proliferation, and Differentiation, Bone Resorption, and the Inhibition Thereof

Methods of measuring osteoclastogenesis, OCL proliferation and differentiation, bone resorption, and the inhibition thereof, are well known in the art. See, e.g., U.S. Pat. Nos. 5,985,832, 5,719,058, 6,093,533, 6,589,528, 5,641,747; Hirayama et al., Effect of corticosteroids on human osteoclast formation and activity. J Endocrinol. 2002 October; 175(1): 155-63; Goldring S R Pathogenesis of bone erosions in rheumatoid arthritis. Curr. Opin. Rheumatol. 2000 May; 12(3): 195-9; Tamura et al., New resorption assay with mouse osteoclast-like multinucleated cells formed in vitro. J Bone Miner Res. 1993 August; 8(8): 953-60; Wei et al., Receptor activator of nuclear factor-kappa b ligand activates nuclear factor-kappa b in osteoclast precursors. Endocrinology. 2001 March; 142(3): 1290-5; Zou et al., Journal of Cellular Biochemistry 83:70-83(2001); Fujikawa et al., The human osteoclast precursor circulates in the monocyte fraction, Endocrinology. 1996 September; 137(9): 4058-60; Takahashi et al., Endocrinology. Osteoclast-like cell formation and its regulation by osteotropic hormones in mouse bone marrow cultures 1988 April; 122(4): 1373-82; Udagawa et al., Origin of osteoclasts: mature monocytes and macrophages are capable of differentiating into osteoclasts under a suitable microenvironment prepared by bone marrow-derived stromal cells. PNAS. 1990 September; 87(18): 7260-4; Crippes et al., Antibody to β3 Integrin Inhibits osteoclast-Mediated Bone Resorption in the Thyroparathyroidectomized Rat, Endocrinology Vol. 137, No. 3 pp. 918-924; and Teitelbaum S L, Bone resorption by osteoclasts. Science. 2000 Sep. 1; 289(5484): 1504-8 (review paper in osteoclasts differentiation and bone resorption). Each of these references are incorporated herein by reference in their entireties. It will be apparent to one skilled in the art that the above-described exemplary methods could be adapted towards making and testing inhibitors of chitinase-like molecules of the invention.

### Example 2

Production of Antibodies Against Chitinase/Chitinase-Like Proteins (C/CLPs)

It is a goal of the present invention to provide inhibitors of C/CLPs, in particular C/CLP inhibitors that reduce the activity of osteoclasts (OCLs) (e.g., inhibit OCL maturation, and/or inhibit OCL differentiation, and/or inhibit osteoclastogenesis). Antibodies or fragments thereof that specifically bind to and inhibit the activity of C/CLPs are preferred inhibitors. Methods for producing antibodies and antibody variants are well known in the art and have been described above. Here we describe the production and characterization of an antibody against recombinant Ym1 protein. In addition, we provide a detailed analysis of the C/CLPs and provide preferred immunogenic domains and epitopes for the production of other inhibitory antibodies, antibody fragments and/or antibody derivatives.

Materials and Methods

Expression and purification of C/CLPs in E. coli: The cloning of recombinant Ym1 (rYml1) into the pET14b expression vector system (Novagen, Inc., Madison, Wis.) was previously described (Oba et al., 2003, J Bone Miner Res.; 18:1332-41). We expressed and purified rYm1 as follows: the BL21 strain of E. coli transformed with the pET14-ECF-L plasmid was grown to an OD₆₀₀ of 0.8 and then gene expression was induced with 1 mM IPTG The culture was allowed to grow for 4 hours before cells were harvested by centrifuigation. The cell pellet was resuspended in 25 ml of 0.5 M NaCl, Tris Cl, pH 8.0 with RNase, DNase and protease inhibitors and then sonicated to lyse the cells. The lysate was clarified by centrifuigation and the pelleted material was washed 2 times with 25 ml of RIPA buffer. The insoluble material was collected after each wash by centrifuigation and then solublized in 25 ml of 8 M Urea, 0.5 M NaCl, 5 mM Imidazole, 20 mM Tris Cl, pH8.0 and applied to a Ni NTA superflow sepharose column. Purified ECF-L was eluted with buffer containing 8 M Urea, 0.5 M NaCl, 250 mM Imidazole, 20 mM Tris Cl, pH8.0. The purified protein was dialyzed against 1×PBS, 25% glycerol.

Cloning of Ym1 for expression in mammalian systems: In order to obtain a version of the Ym1 gene which contained a 6× Histidine tag at the carboxy terminus, the Ym1 gene was amplified by PCR using mouse cDNA (Clontech), a forward primer (table 1) containing a BamHI site (bolded) and a Kozak consensus (underlined) up stream of the ATG codon, and a reverse primer (table 1) containing an EcoRI (bolded) site distal to the stop codon (italics) and sequence coding for 6 histidine residues (underlined) 5′ to the last naturally occurring codon. The PCR product was digested with BamHI and EcoRI and cloned into pcDNA3.1. The sequence of the clone was confirmed by DNA sequence analysis.

Cloning of Ym2, mouse AMCase, and mouse chitotriosidase for expression in mammalian systems: The construction of histidine tagged versions of Ym2, mouse AMCase and mouse chitotriosidase was accomplished by utilizing PCR in a similar manner as the Ym1 using mouse cDNA as template, a forward primer with a convenient restriction site, a Kozak consensus sequence upstream of the initiation codon, and a reverse primer that inserted 6 histidine codons between the last naturally occurring codon and the stop codon, followed by a convenient restriction site (see table 1 for full description of all primers). After amplification, the PCR products were digested with restriction endonucleases and ligated into pcDNA3.1. The template used for amplification of the mouse AMCase gene was a cDNA clone purchased from Open Biosystems. These genes were cloned with a BamHI site at the amino terminus and an EcoRI site at the carboxy terminus except for mouse chitotriosidase construct, which in order to circumvent an internal BamHI site, utilized a Bgl II site at the amino terminus.

TABLE 1
Primers used for cloning and construction of polynucleotides encoding C/CLPs

| | | |
|---|---|---|
| Name of | | |
| gene | Forward primer | Reverse primer |

| | | |
|---|---|---|
| Ym1 | 5′GGATCCCAACATGGGCAAGCTCATTCTTGTC3′ | 5′GAATT [symbol] TAATGGTGATGGTGATGGTGATAAGGGCCCTTGCAACT3′ |
| | SEQ ID NO:1 | SEQ ID NO:5 |
| Ym2 | 5′GGATCCCAACATGGCCAAGCTCATTCTTTGTC3′ | 5′GAATT [symbol] TAGTGATGGTGATGGTGATGAAGCTCCCCTCGATAAGAGGC3′ |
| | SEQ ID NO:2 | SEQ ID NO:6 |
| Mouse | 5′GGATCCCACCATGGCCAAGCTACTTCTCGTC3′ | 5′GAATT [symbol] TAGTGATGGTGATGGTGATGTGGCCAGTTGCAGCAATT3′ |
| AMCase | SEQ ID NO:3 | SEQ ID NO:7 |
| Mouse | 5′AGATCTCACCATGGTGCAGTCCCTGGCCTGG3′ | 5′GAATT [symbol] TAGTGATGGTGATGGTGATGGACTGGAGTTGGATGGGG3′ |
| chito- | SEQ ID NO:4 | SEQ ID NO:8 |
| triosidase | | |

Forward primer: restriction endonuclease site-bolded, Kozak consensus-underlined.
Reverse primer: restriction endonuclease site-bolded, stop codon- italics, 6 histidine tag- underlined.

Expression of C/CLPs in mammalian cells: HEK293 cells are transformed using the FreeSyle 293 Expression System for large scale transfections (InVitrogen, Carlsbad, Calif.). Briefly, 450 micrograms of plasmid is mixed with 200 microliters of 293fectin diluted in a final volume of 30 ml Optimem. This is added to a 450 ml culture of 293 cells growing in 293 FreeStyle Media. The culture supernatant is harvested 3 days after transfection, and the cells are fed. A second harvest is done after an additional 3 days. The protein is then purified by applying the culture supernatant to a Nickel NTA sepharose column. The protein is eluted with an Imidazole gradient.

Generation and Characterization of polyclonanal antisera against rYm1: Polyclonal antibodies to Ym1 were generated by immunizing rabbits with purified rYm1, expressed and purified from E. coli, using methods well known in the art and described in, for example, Harlow et al. (1989, Antibodies: A laboratory Manual, Cold Spring Harbor, N.Y.).

Antiserum from rabbit #2870 (referred herein as anti-Ym1 serum) was titered by ELISA on several recombinant chitinase molecules using standard procedures. The endpoint titer was defined as the greatest dilution that yielded an OD value greater then twice that achieved with the corresponding pre-bleed serum sample from the same animal.

Results

The results of the ELISA assay summarized in table 2 demonstrate that the antisera against Ym1 from rabbit #2870 strongly recognizes both Ym1 and the very highly related (nearly 92% identical) Ym2. We see some cross reactivity with other members of the C/CLP family. This result is not unexpected, from a polyclonal anti-sera raised against a whole protein, given the high degree of homology among the C/CLP family members (see FIGS. 1a, 1b and discussion below). A more specific antibody can be generated by any number of standard techniques including generation of monoclonal antibodies and/or the use of specific peptides as antigens for immunization.

TABLE 2
Characterization of anti-Ym1 antisera
ANTIGEN

| Ym1 | Ym2 | mAMCase | mchitotriosidase |
|---|---|---|---|
| 1:2,560,000 | 1:2,560,000 | 1:64,000 | 1:32,000 |

Antiserum from rabbit #2870 was tittered by ELISA on the indicated recombinant proteins (mouse AMCase and mouse chitotriosidase).

To better understand the observed cross reactivity pairwise comparisons of Ym1 with the mouse AMCase and the mouse chitotriosidase were performed using the MegaAlign program (DNASTAR) with the Clustal W algorithm (Thompson et al., 1994 Nucleic Acids Res 22:4673-80). The results (FIG. 1A) show that there are multiple regions each protein that share a high degree of similarity with Ym 1. A similar analysis of Ym1 and the human YKL-39, YKL-40, AMCase and chitotriosidase was performed. The results, shown in FIG. 1B, indicate that YKL-39 is most closely related to YKL-40 followed by chitotriosidase, AMCase and Ym1. There is at least 53% identity between any two of the C/CLPs, and a much higher percent similarity.

To identify potential immunogenic peptides for use in generating antibodies that could be either protein specific or would bind with one or more C/CLPs, the antigenic index of each protein was examined using the Protean program (DNASTAR) with the Jameson-Wolf algorithm. The regions with the highest antigenic indices among all members of the C/CLP family are indicated with the double underline in FIG. 1B. It is readily apparent that many of these regions are highly conserved among one or more family members and would be excellent candidates for raising an antibody which recognizes more then one family member. While the use of less conserved regions would likely generate an antibody specific for one C/CLP family member.

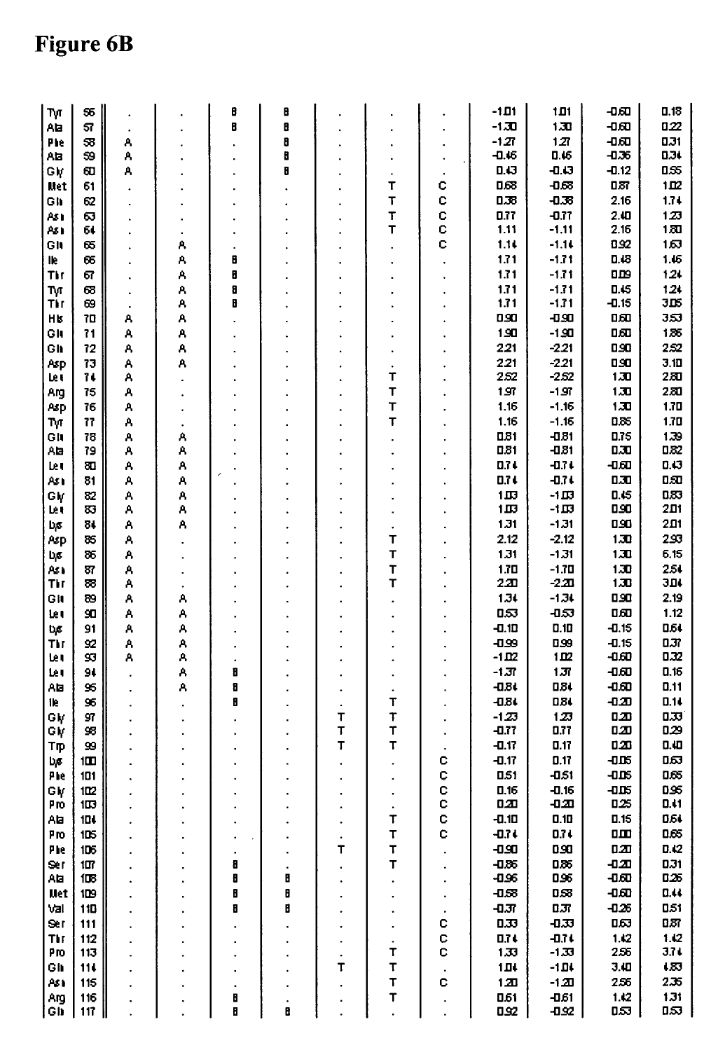

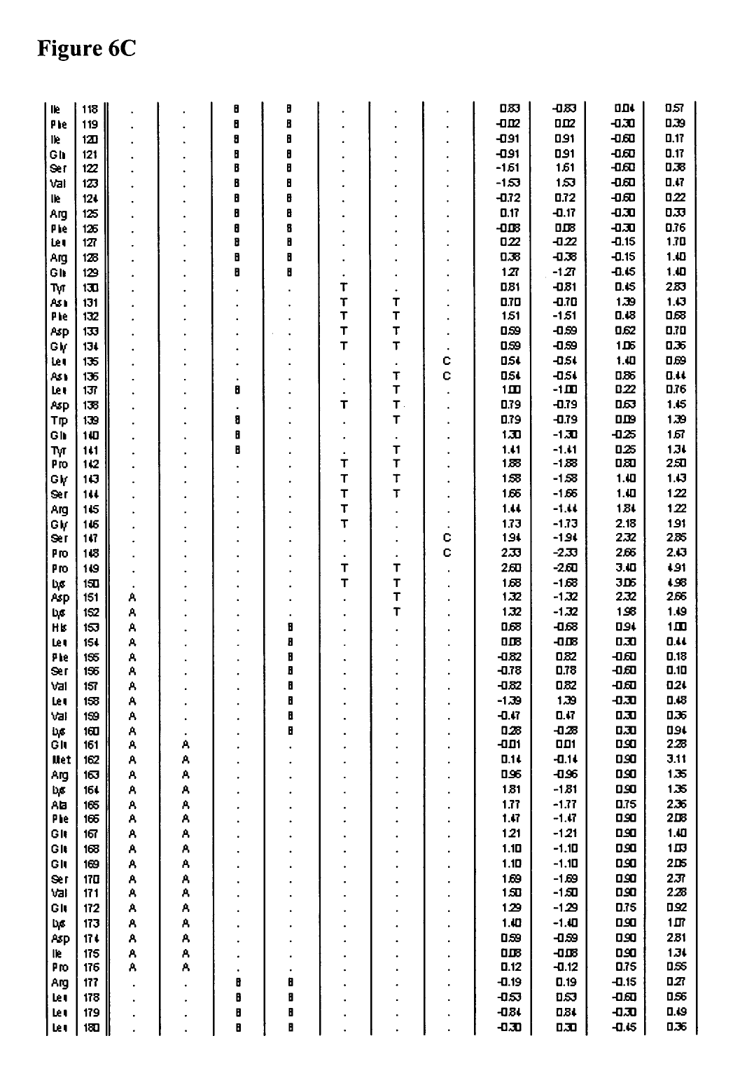

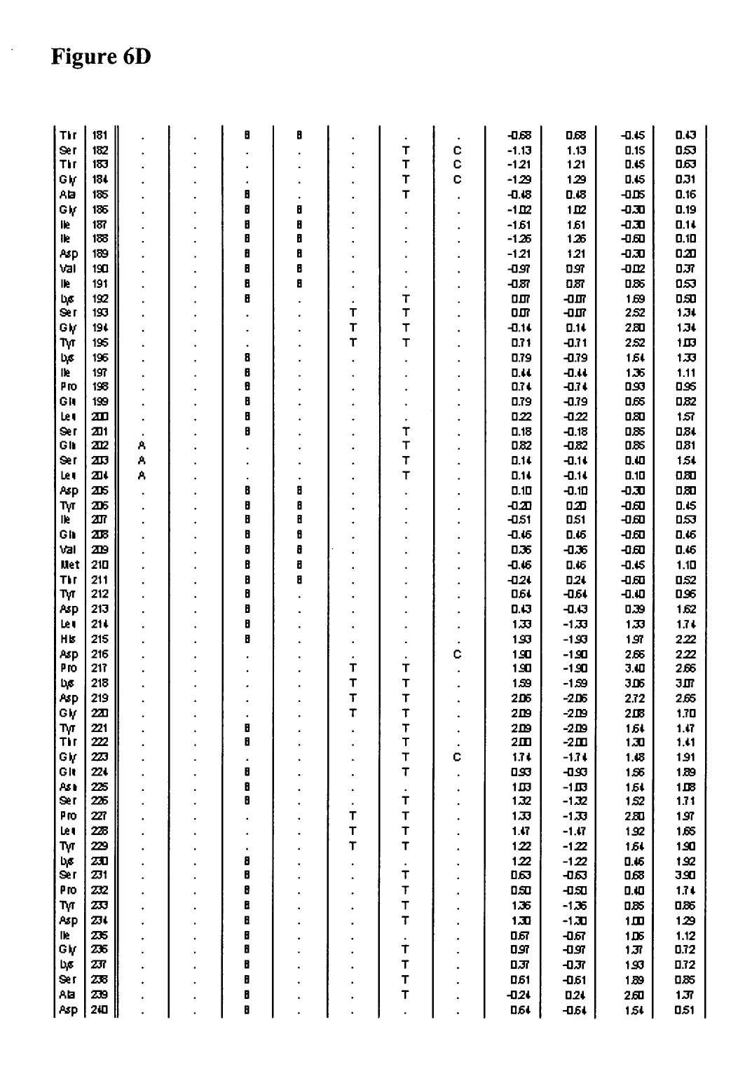

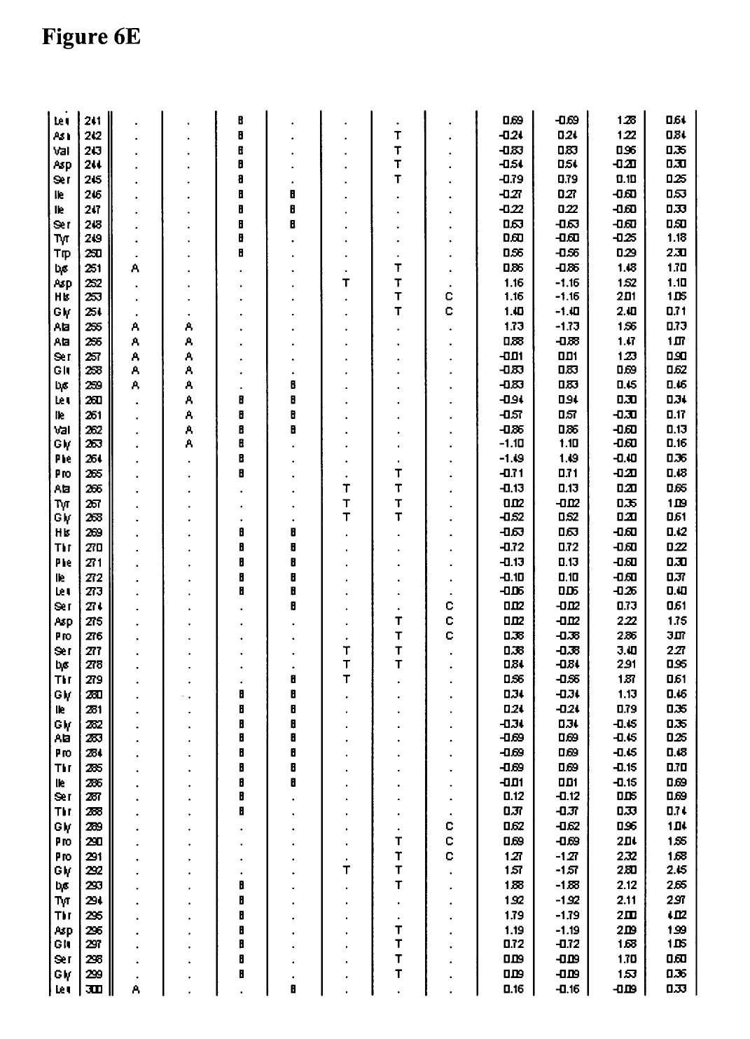

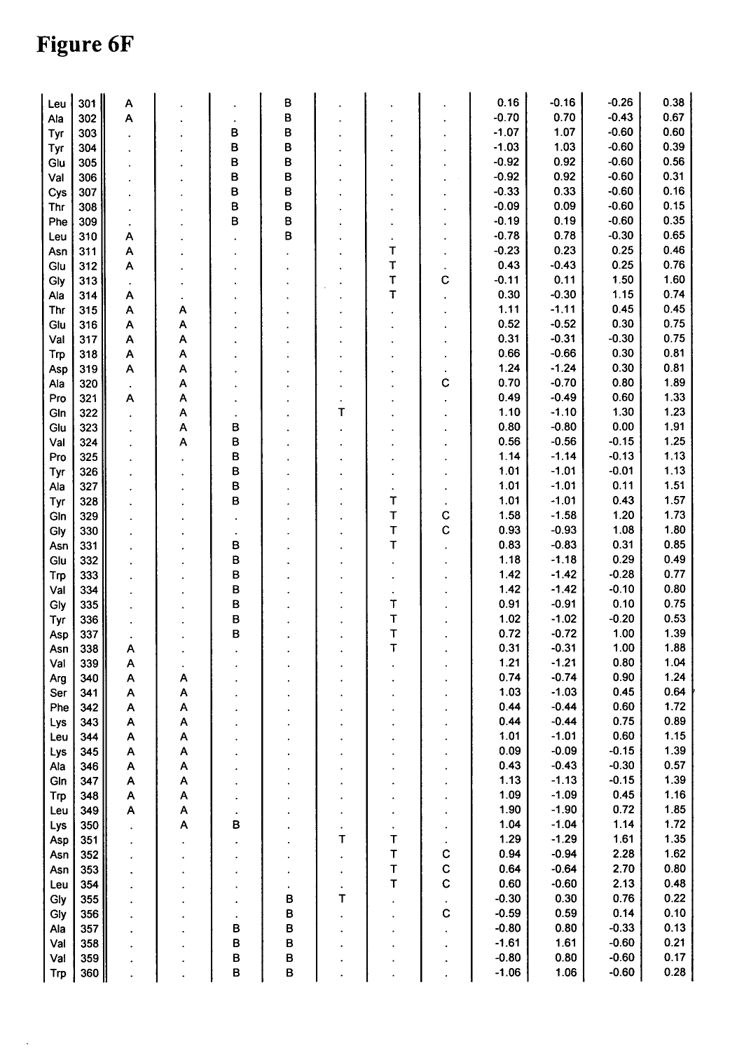

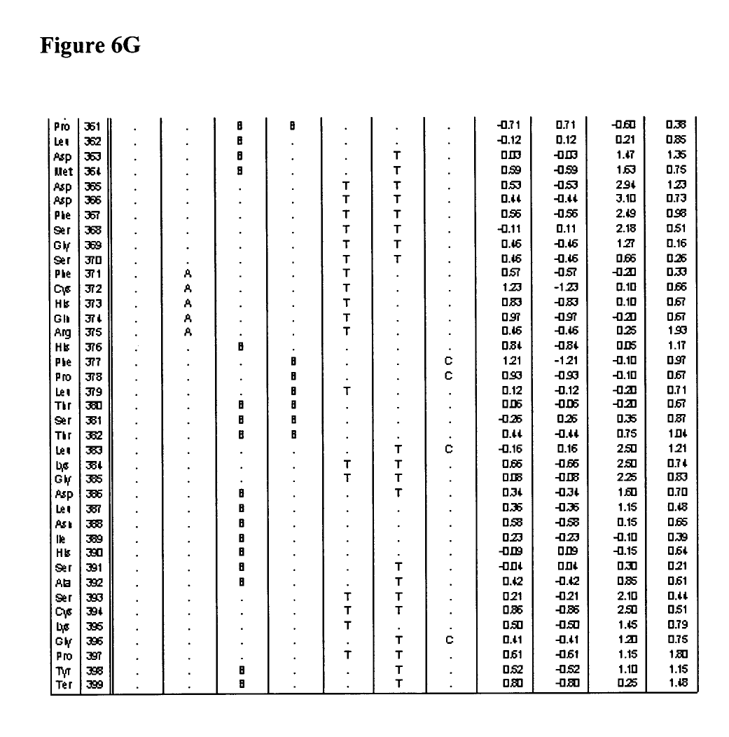

Amore detailed analysis of the protein structure and characteristics of Ym1 were performed using several well-established algorithms. The plotted data is shown in FIG. 1C. The calculated values for each amino acid are found in FIGS. 6A-G From these data it would be readily apparent to one skilled in the art, which regions of the protein would be most antigenic and which regions could be of structural and/or functional relevance. A similar analysis can be readily performed for any member of the C/CLP family of proteins using commercially available software such as DNASTAR.

### Example 3

Ym1 is Present in the Inflamed Joints of Collagen-Induced Arthritis (CIA) Mouse Model.

It had previously been reported that C/CLPs are up regulated and/or associated with osteloclast-related conditions (see e.g., Oba et al., 2003 J Bone Miner Res.; 18:1332-41; Knorr et al., 2003, Ann Rheum Dis 62:995-8; Johansen et al., 1993 British J of Rheum 32:949-55, Hakala et al., 1993 J Biol Chem 268:25803-10). The type II collagen-induced arthritis (CIA) mouse has been extensively used as a murine model for, the human osteloclast-related condition, rheumatoid arthritis (RA). The disease is characterized by severe synovial inflammation that results in destruction of joint tissues and cartilage/bone erosions (see e.g., Stuart et al., 1984 Annu. Rev. Immunol. 2:199-218; Trentham et al., 1982 Arthritis Rheum. 25:911-6; Trentham et al., 1977 J Exp Med 146:857-68). We demonstrated that Ym1 is present in the inflamed joints of CIA mice by immunohistochemical staining.

Materials and Methods

Induction of Collagen Induced Arthritis (CIA): Six-eight week old male DBA/1LacJ mice (Jackson Labs, Bar Harbor, Me.) were used. On day 0, isoflurane anesthetized animals were given an intradermal injection, at base of tail, of 200 μg bovine Type II collagen (CII) dissolved in 50 μl 0.05 M acetic acid and mixed with an equal volume of complete Freund's adjuvant (Chondrex, Redmond, Wash.). Three weeks later on day 21 they were given a second similar intradermal injection of 100 μg of CII dissolved in 25 μl 0.05 M acetic acid and mixed with an equal volume of in incomplete Freund's adjuvant (Difco, Detroit, Mich.).

Immunohistochemistry: Formalin fixed paraffin embedded tissue sections of mouse joints were mounted on glass microscope slides and stained with an anti-Ym1 Ab. Quenching of endogenous peroxidase was performed by incubating tissue sections with 3% H₂O₂ at RT for 10 minutes. Tissue sections were treated with blocking solution (5% BSA/TBS+Tween) at RT for 30 minutes, followed by incubation with anti-Ym1 at 1 μg/ml, in humidity chamber, over night at RT. Sections were then washed and treated with the Biotinylated goat-anti-rabbit IgG (Dako, Carpinteria, Calif.) at 2 μg/ml for at RT for 10 minutes. Slides were then washed again and HRP-conjugated Streptavidin (Dako) diluted to 1.6 μg/ml was applied at RT for 30 minutes. The biotinylated goat-anti-rabbit and HRP-conjugated Streptavidin incubations were repeated. Slides were incubated with a DAB staining kit (Sigma, St. Louis, Mo.) for 4 minutes for color visualization. Slides were counterstained by incubation with Myers Hematoxylin for 2 minutes.

Results

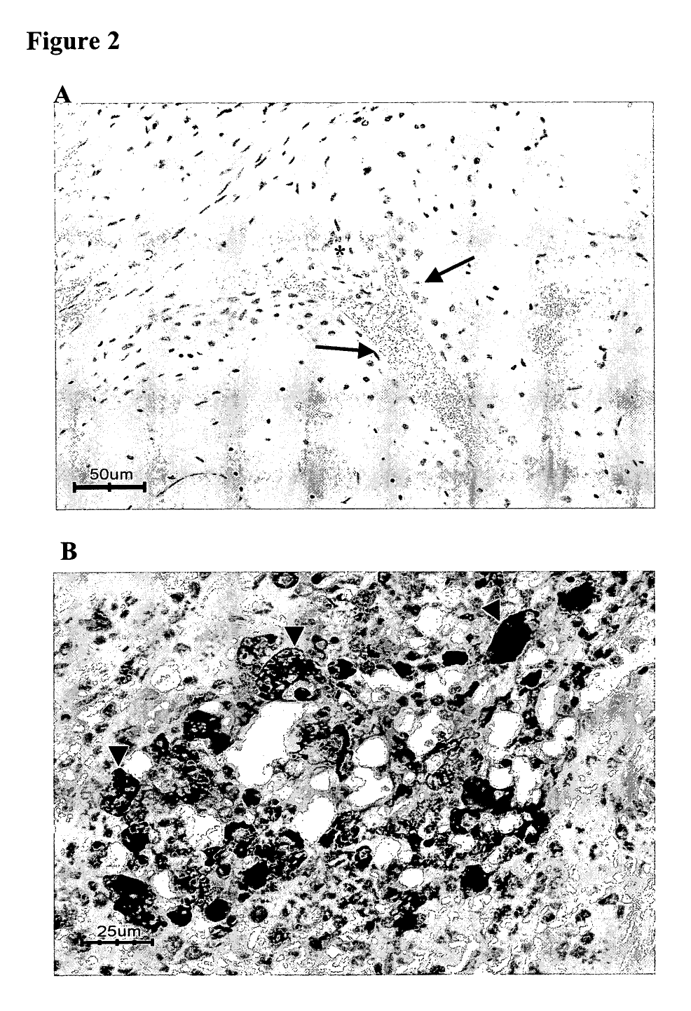

To begin to address the role of C/CLPs in vivo, we examined the joints of normal and CIA mice for the presence or absence of Ym1 immunohistochemistry using an anti-Ym1 antibody. We found that Ym1 was virtually undetectable in the joints of a normal animal (FIG. 2A). However, staining increased dramatically in the affected joints of CIA mice (compare FIGS. 2A and 2B). The most intense staining was seen in the areas of active inflammation and tissue damage. Macrophages (arrow heads in FIG. 2B) are clearly present in the inflamed areas. A negative control antibody showed no staining in the joints of CIA mice (data not shown). Since by ELISA we found that the anti-Ym1 antibody also binds the very highly related Ym2 protein we cannot eliminate the possibility that both Ym1 and Ym2, or possibly Ym2 alone is being up-regulated in the joints of the CIA mouse. This question can readily be addressed by the use of RT-PCR to examine the expression of the two molecules in the joints of normal versus CIA mice.

### Example 4

Administration of an Anti-Ym1 Antibody Significantly Reduces the Severity of Disease Progression in the Collagen-Induced Arthritis (CIA) Mouse Model.

To determine if an antibody (or another antagonist) against a C/CLP was a useful therapeutic we used our anti-Ym1 antibody to treat type II collagen-induced arthritis (CIA) in a mouse model. Two different treatment protocols may be used with this model to assess the effect of a given therapy: 1) prevention model in which treatment is initiated prior to collagen immunization, and 2) therapeutic model in which treatment is initiated after the onset of clinical arthritis. We demonstrate here, for the first time, that an antibody against a C/CLP (Ym1) demonstrated efficacy in both prevention and therapeutic RA models. In addition, we show for the first time, in an in vivo model, that the C/CLP are valid therapeutic targets for the inhibition of osteoclast-related illness.

Treatment Protocols for Mice Immunized with CII: DBA/1LacJ male mice (6-8 weeks old) were divided into 4 groups of 6 mice/group and treated as described above to induce arthritis. For the protection model rabbit anti-Ym1 serum was administered as an intraperitoneal injection at day 20, 22, 24 and 26. The anti-Ym1 treated group received 0.5 ml per animal per dose. A second group received normal control rabbit sera (0.5 ml per animal). The third group received mouse IgG (10 mg/kg at day 24, 26, 28 and 30) as a negative treatment control, while the forth group was untreated.

For the therapeutic model rabbit anti-Ym1 serum was administered as an intraperitoneal injection at the onset of inflammation, day 21, and continued daily until day 28. The anti-Ym1 treated group received 0.2 ml per animal per dose as did the second group receiving control normal rabbit sera. A third group received PBS as a negative treatment control, while the fourth group was untreated.

For both studies the extent of disease was evaluated in each paw and assigned a clinical score (0-3) to each paw: 0=Normal, 1=definite swelling, 2=severe swelling and 3=maximal swelling. The lateral digits are also scored and a final clinical score is determined as detailed below.

Monitoring Disease: Beginning on the day of the CII boost (day 21) all animals were observed daily to assess the status of the disease in their paws, which was done by assigning a qualitative clinical score to each of the paws. Every day, each animal has its 4 paws scored according to its state of clinical disease. The scoring is done by two observers, with at least one blinded. In addition, each mouse is weighed, to follow body weight changes, at the same time the paws are scored.
Grading scales for the ankle/wrist/midfoot/forfoot are as follows:
0=Normal
1=definite swelling
2=severe swelling
3=maximally severe swelling and non-weight bearing

The grading scale for the 4 lateral digits of each are graded as involved or not involved, i.e., 1 or 0. For example, a maximally involved left rear paw would be scored: ankle=3, midfoot=3, digits=4 (clinical score=10 units) We would repeat this for each paw and sum the scores.

Mice are euthanized at day 32, or sooner if total clinical score reaches 40 and a histological evaluation of joints is performed.

Histology: Hind-limb tibiotalus joints from each animal were evaluated and scored for histologic changes as described in Badger et al., 2001, Arthritis & Rheumatism 44:128-37. Briefly, animals were sacrificed on day 32 and the hind legs were fixed in formaline and decalcified in Cal-Rite (Richard-Allen Scientific, Kalamazoo, Mich.). The paws were then removed from the legs at the distal tibial diaphysis. After routine processing, the samples were embedded and coronal sections were cut in the plane midway through the tibiotalus and talartarsal joints. Sections were stained with Safranin O and counterstained with fast Green.

The histological characteristic of the bone and articular cartilage/periarticular soft tissue were scored separately by a blinded observer. The bone was graded as follows: 0=normal, 1=subperiosteal fibrosis with periosteal woven bone formation, 2=Marrow inflammation, endosteal and trabecular bone resorption, 3=extensive inflammation, 4=marrow replaced by granulation tissue, little trabecular bone remaining, extensive obliteration of cortical contours. The cartilage/synovium was graded as follows: 0=normal, 1=mild lymphocytic inflammation in synovium and surrounding tissues, 2=synovial fibrosis and edema, partial lymphocytic infiltration of the joint space, minor pannus erosion of the cartilage, 3=extensive infiltration of joint space, peripheral and subchondral cartilage erosion, extensive fibrosis of soft tissue with regional necrotic liquefaction.

Results

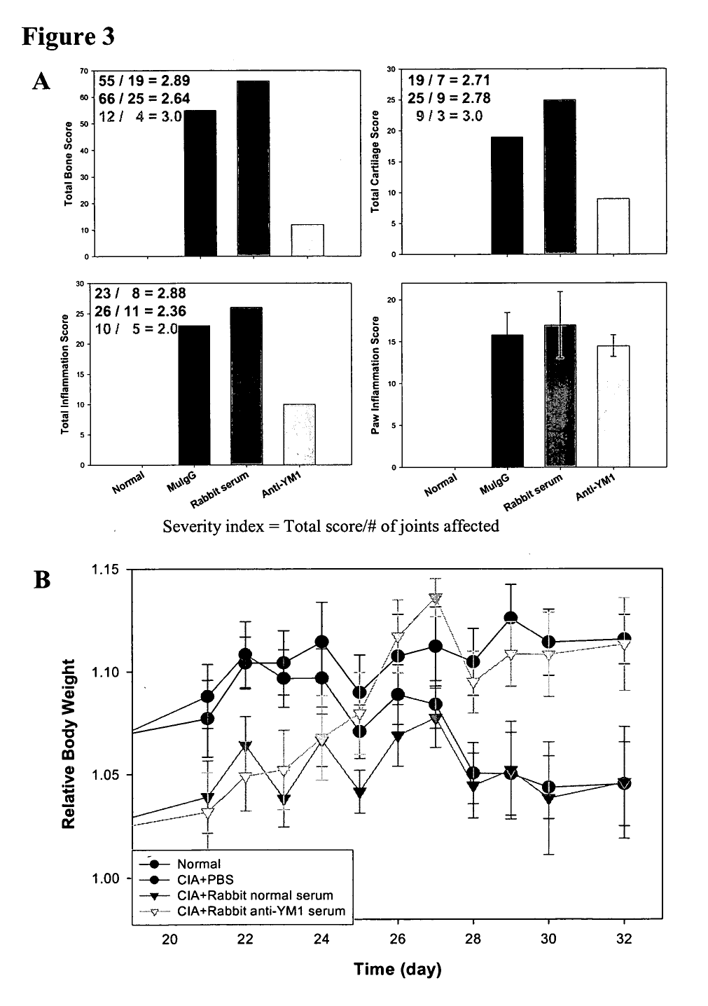

To demonstrate whether treatment with Ym1 antibodies could prevent CIA. Mice were treated with anti-Ym1 sera (0.5 ml/mouse) starting one day before mice had been immunized with collagen. Mice received four doses over a six day period. At the same time control mice (6 mice/group) received either normal rabbit serum (0.5 ml/mouse) or mouse IgG (10 mg/kg). The development of arthritis was assessed daily for 12 days after the initial treatment. The graphs in FIG. 3A show the total clinical scores of the observed paw inflammation as well as the total histological scores for bone, cartilage and inflammation for the prevention model of CIA. The administration of normal rabbit serum or mouse IgG had no effect on the development of arthritis. However, the anti-Ym1 treated animals have greatly reduced bone (>4.5 fold decrease), cartilage (>2.0 fold decrease) and total inflammation (>2.3 fold decrease) scores compared to the control animals. Another clinical feature of disease progression is weight loss. The relative body weight scores for the control animal show a net decrease during the course of the study while the anti-Ym1 animals show a net increase in their relative body weight similar to that seen in the normal animals (FIG. 3B). These results demonstrate that anti-Ym1 antibodies can effectively protect against joint damage and other symptoms when administered prior to the onset of disease.

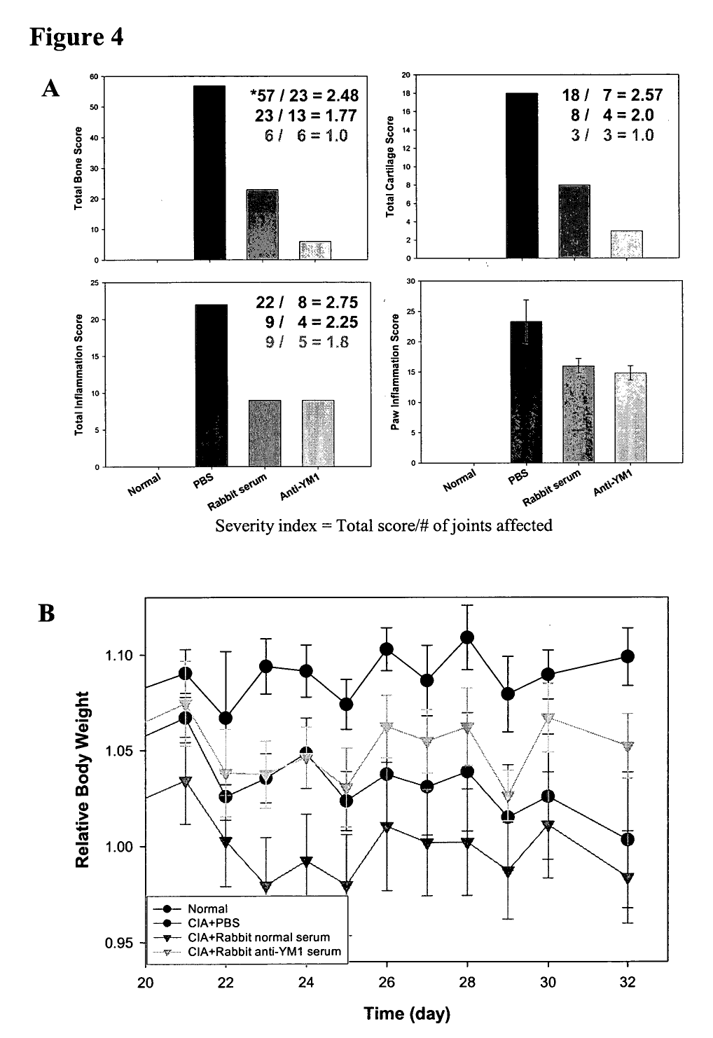

An even more striking and clinically relevant result was the dramatic efficacy seen in the therapeutic model. In this model the anti-Ym1 was administered (0.2 ml/mouse) after the onset of disease symptoms. Again a dramatic reduction in both the bone (>3.8 fold decrease) and cartilage (>2.6 fold decrease) scores were seen in the Ym1 treated animals compared to the control rabbit serum or PBS treated animals (FIG. 4A). Although the decrease in the total inflammation scores were less impressive this is to be expected because in this model treatment is not started until the onset of observable symptoms namely, paw inflammation. While the anti-Ym1 treated group did not show a significant weight gain, they did maintain their relative body weight unlike the control treated group, which showed a net decrease in their relative weight (FIG. 4B).

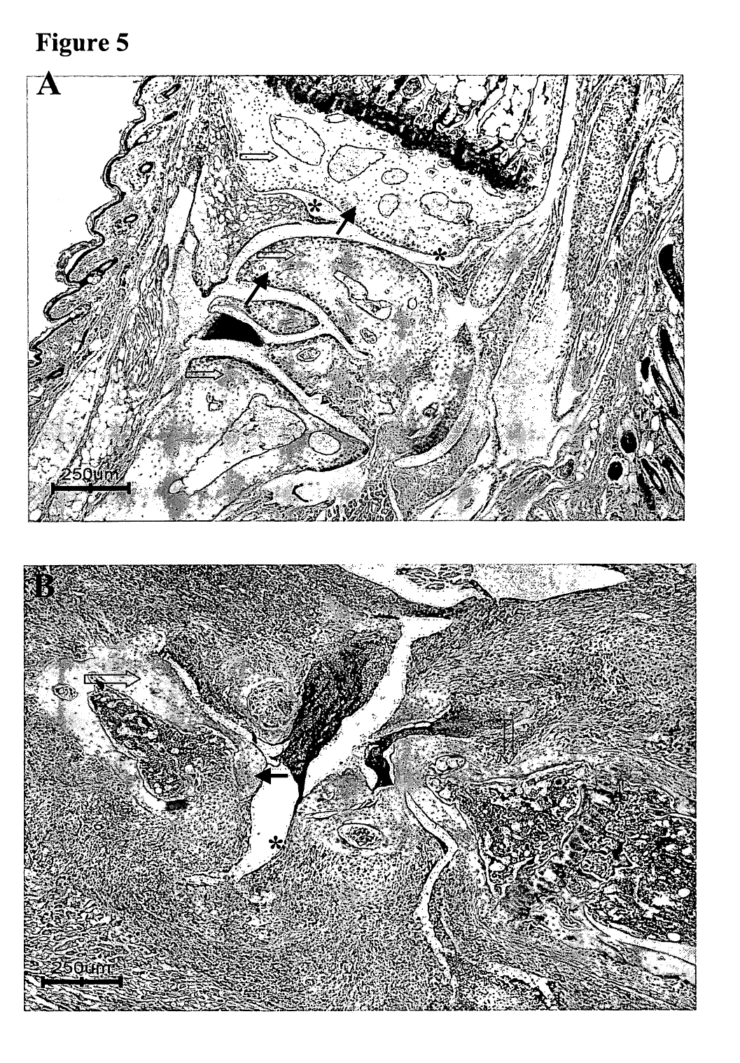

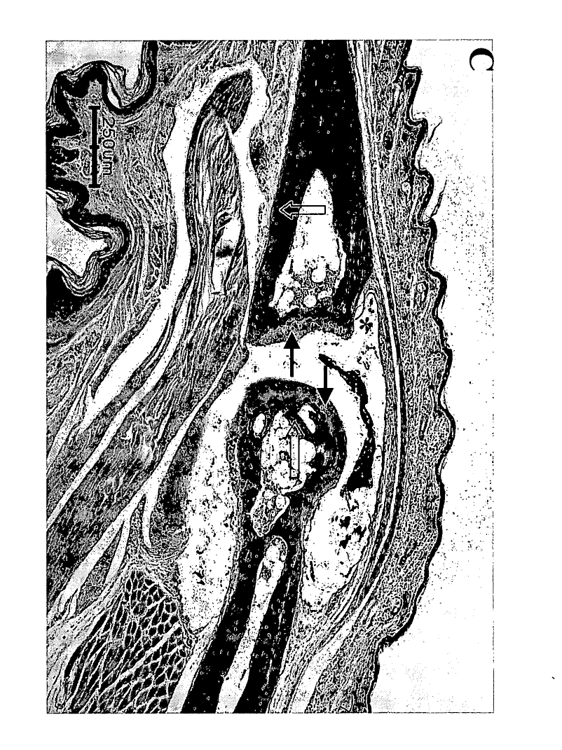

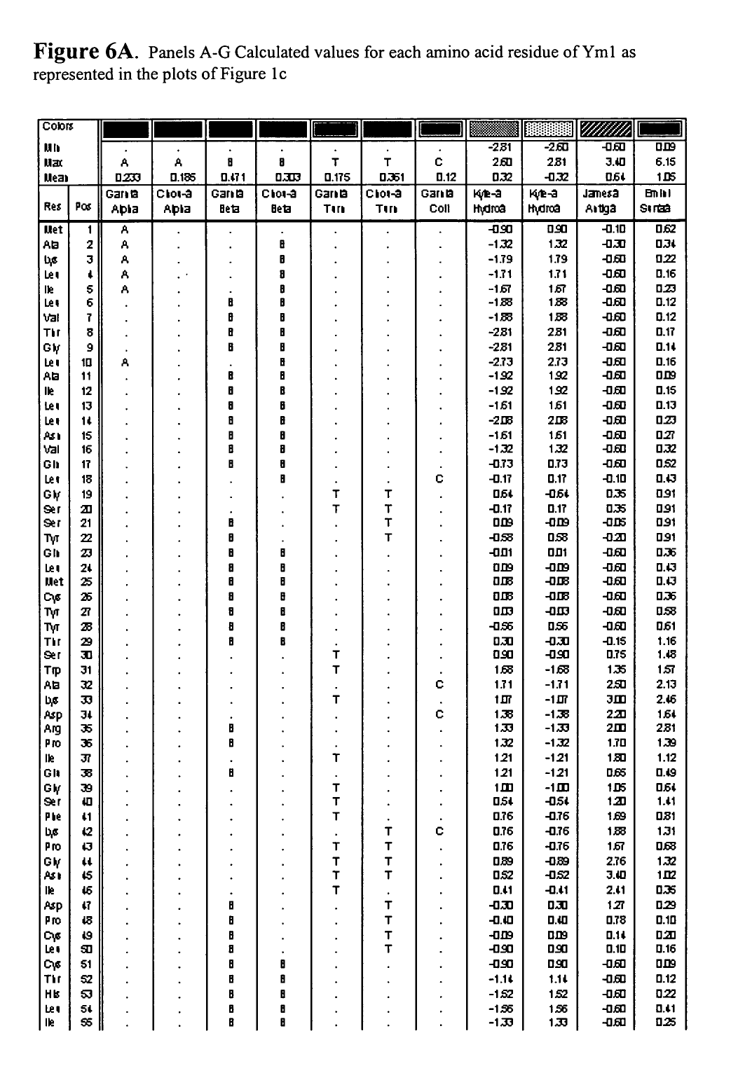

The dramatic effects of the anti-Ym1 treatment on disease manifestation and progression can also be seen by histological examination of joints. FIG. 5a shows a representative joint from normal animal. FIG. 5B is a joint from a control PBS treated animal from the therapeutic model study. As expected all the histologic changes of severe arthritis were exhibited with nearly all the joints showing pronounced synovial hyperplasia, cartilage erosion, massive infiltration of inflammatory cells and bone deterioration. In stark contrast the Ym-1 treated animal (FIG. 5C) has few inflammatory cells present and the bone and cartilage are nearly intact. These results indicate that anti-Ym1 treatment had a profound effect in diminishing the disease severity in CIA mice even when administered therapeutically after the onset of symptoms.

### Discussion

Although the up regulation and/or accumulation of certain C/CLPs has been well correlated with joint disease and/or injury, a mechanistic link had not been well established. The recent observation that Ym1 is a co-stimulatory molecule, enhancing the later stages of OCL formation in tissue culture, suggests that the chitinase-like proteins, Ym1 in particular, may play an active role in OCL maturation and/or connective tissue damage and inflammation. OCLs have recently been show to be the essential link between synovial inflammation and bone loss (Redlich et al., 2002 J Clin Invest 110:1419-27). Thus, a treatment that can inhibit osteoclast differentiation and/or activation changes a destructive arthritis to a nondestructive form. As mediators in OCL maturation, the chitinase-like molecules, Ym1 in particular, may play a key role in the development and progression of joint disease. As such, Ym1 and/or other chitinase-like molecules are ideal targets for the development of therapeutics to treat inflammatory joint diseases. Such a therapy will be of significant clinical benefit to patients who suffer from RA or other osteoclast-related disorders.

We generated antibodies against Ym1 and found that that, like several other C/CLPs, Ym1 levels are dramatically up regulated in the joints of CIA mice. The correlation of Ym1 staining with disease progression in a mouse model supports the fact that Ym1 acts as a mediator of RA by stimulating OCL maturation. These results also suggest that the C/CLP family of molecules, Ym1 and it's orthologues in particular, could be effective targets for the treatment of RA and other inflammatory joint diseases or injuries.

Next we examined the effects of anti-Ym1 antibody treatment in vivo using the well-established CIA mouse model of RA. We demonstrated, for the first time, that anti-Ym1 treatments had significant prevention and therapeutic efficacy in the CIA mouse model. However, the polyclonal anti-Ym1 is seen to bind to a lesser extent with mouse AMCase and mouse chitotriosidase, both of which have been identified as markers of inflammation. Thus, the data demonstrate that targeting one or more of these proteins with an antibody-based treatment is highly efficacious in protecting a mammal from the pathological consequences of CIA induced RA. Combinations of specific antibodies could be utilized that would inhibit both OCL maturation and inflammatory responses mediated by the C/CLP family of molecules. In summary, we have demonstrated that inhibiting at least one C/CLP, for example Ym1, can provide clinical benefits for the treatment of RA. These results suggest that similar therapies can be extended to treat other inflammatory joint diseases and OCL-related disorders including bone metabolism and connective tissue disorders and diseases.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. The appended claims are intended to be construed to include all such embodiments and equivalent variations.

All publications, patents, patent applications, or other documents cited in this application are incorporated by reference in their entirety for all purposes to the same extent as if each individual publication, patent, patent application, or other document were individually indicated to be incorporated by reference for all purposes.

1. A method of inhibiting synovial hyperplasia, cartilage damage, infiltration of inflammatory cells and bone deterioration associated with an arthritic disease in a mammal comprising inhibiting the expression or activity of one or more C/CLP.

2. The method of claim 1, further comprising inhibiting TNF-α expression or activity.

3. The method of claim 1 further comprising inhibiting RANKL expression or activity.

4. The method of claim 1, 2 or 3, wherein an isolated antibody or fragment thereof which specifically binds one or more C/CLP, inhibits the expression or activity of one or more C/CLP.

5. A method of inhibiting synovial hyperplasia associated with an arthritic disease in a mammal comprising administering an isolated antibody or fragment thereof that specifically binds one or more C/CLP.

6. A method of inhibiting cartilage damage associated with an arthritic disease in a mammal comprising administering an isolated antibody or fragment thereof that specifically binds one or more C/CLP.

7. A method of inhibiting infiltration of inflammatory cells associated with an arthritic disease in a mammal comprising administering an isolated antibody or fragment thereof that specifically binds one or more C/CLP.

8. A method of inhibiting bone deterioration associated with an arthritic disease in a mammal comprising administering an isolated antibody or fragment thereof that specifically binds one or more C/CLP.bone.

9. The isolated antibody of fragment thereof of claims 5, 6, 7, or 8 wherein said antibody specifically binds one or more C/CLP selected from the group consisting of:
a) ECF-L;
b) acidic mammalian chitinase;
c) Ym1;
d) Ym2;
e) oviductal glycoprotein 1;
f) cartilage glycoprotein 1;
g) chitotriosidase;
h) oviductal glycoprotein 1;
i) cartilage glycoprotein-39;
j) chondrocyte protein 39;
k) TSA1902-S; and
l) TSA1902-L.

10. A method of treating or preventing a connective tissue disease, comprising administering to a human a biologically effective amount of an isolated antibody of fragment that specifically binds one or more C/CLP selected from the group consisting of:
m) ECF-L;
n) acidic mammalian chitinase;
o) Ym1;
p) Ym2;
q) oviductal glycoprotein 1;
r) cartilage glycoprotein 1;
s) chitotriosidase;
t) oviductal glycoprotein 1;
u) cartilage glycoprotein-39;
v) chondrocyte protein 39;
w) TSA1902-S; and
x) TSA1902-L.

11. The method of claim 10, wherein said C/CLP is of human origin.

12. The method of claim 10, wherein said connective tissue disease or disorder is selected from the group consisting of:
a) Paget's disease;
b) osteoporosis;
c) Gorham-Stout syndrome;
d) arthritis;
e) osteoarthritis;
f) rheumatoid arthritis;
g) psoriatic arthritis; and
h) brittle bone disease.

13. An isolated antibody or fragment thereof that specifically binds to a polypeptide at least 90% identical to Ym1, wherein said antibody or fragment thereof inhibits inflammation or bone deterioration associated with an arthritic disease when administered to a mammal.

14. The isolated antibody or fragment thereof of claim 13, wherein said antibody or fragment thereof specifically binds to a polypeptide at least 92% identical to Ym1.

15. The isolated antibody or fragment thereof of claim 13, wherein said antibody or fragment thereof specifically binds to a polypeptide at least 95% identical to Ym1.

16. The isolated antibody or fragment thereof of claim 13, wherein said antibody or fragment thereof specifically binds to a polypeptide at least 99% identical to Ym1.


SEQUENCE LISTING (16 sequences)

SEQ ID NO: 1 - DNA, 31 bp, Artificial
  synthetic primer used for construction of vectors
ggatcccaacatggccaagctcattcttgtc

SEQ ID NO: 2 - DNA, 31 bp, Artificial; identical to SEQ ID NO: 1
  Synthetic primer

SEQ ID NO: 3 - DNA, 31 bp, Artificial
  Synthetic primer
ggatcccaccatggccaagctacttctcgtc

SEQ ID NO: 4 - DNA, 31 bp, Artificial
  Synthetic primer
agatctcaccatggtgcagtccctggcctgg

SEQ ID NO: 5 - DNA, 44 bp, Artificial
  Synthetic primer
gaattctaatggtgatggtgatggtgataagggcccttgcaact

SEQ ID NO: 6 - DNA, 47 bp, Artificial
  Synthetic primer
gaattctagtgatggtgatggtgatgaagctcccctcgataagaggc

SEQ ID NO: 7 - DNA, 44 bp, Artificial
  Synthetic primer
gaattctagtgatggtgatggtgatgtggccagttgcagcaatt

SEQ ID NO: 8 - DNA, 44 bp, Artificial
  Synthetic primer
gaattctagtgatggtgatggtgatggactggagttggatgggg

SEQ ID NO: 9 - protein, 398 aa, Mus musculus
MAKLILVTGLAILLNVQLGSSYQLMCYYTSWAKDRPIEGSFKPGNIDPCLCTHLIYAFAGMQNNEITYTHEQDLRDYEALNGLKDKNTELKTLLAIGGWKFGPAPFSAMVSTPQNRQIFIQSVIRFLRQYNFDGLNLDWQYPGSRGSPPKDKHLFSVLVKEMRKAFEEESVEKDIPRLLLTSTGAGIIDVIKSGYKIPELSQSLDYIQVMTYDLHDPKDGYTGENSPLYKSPYDIGKSADLNVDSIISYWKDHGAASEKLIVGFPAYGHTFILSDPSKTGIGAPTISTGPPGKYTDESGLLAYYEVCTFLNEGATEVWDAPQEVPYAYQGNEWVGYDNVRSFKLKAQWLKDNNLGGAVVWPLDMDDFSGSFCHQRHFPLTSTLKGDLNIHSASCKGPY

SEQ ID NO: 10 - protein, 402 aa, Mus musculus
MAKLILVTGLAILLNVQLGSSYQLMCYYTSWAKDRPTEGSFKPGNIDPCLCTHLIYAFAGMKNNEITYLSEQDLRDYEALNGLKDRNTELKTLLAIGGWKFGPAPFSSMVSTPQNRQTFIKSVIRFLRQYNFDGLNLDWQYPGSRGSPPKDKHLFSVLVQEMRKAFEEESTLNHIPRLLLTSTGAGFIDVIKSGYKIPELSQSLDYIQVMTYDLHDPKNGYTGENSPLYKSPYDIGKSADLNVDSIITYWKDHGAASEKLIVGFPAYGHTFILSDPSKNGIGDPTVSAGPPGKYTNEQGLLAYFEICTFLNEGATEIFDATQEVPYAYLGNEWVGYDNVRSFKLKAQWLKDNNLGGAVVWPLDMDDFSGSFCHQGRFPLTTTLKRDLNVHSASCKASYRGEL

SEQ ID NO: 11 - protein, 473 aa, Mus musculus
MAKLLLVTGLALLLNAQLGSAYNLICYFTNWAQYRPGLGSFKPDDINPCLCTHLIYAFAGMQNNEITTIEWNDVTLYKAFNDLKNRNSKLKTLLAIGGWNFGTAPFTTMVSTSQNRQTFITSVIKFLRQYGFDGLDLDWEYPGSRGSPPQDKHLFTVLVKEMREAFEQEAIESNRPRLMVTAAVAGGISNIQAGYEIPELSKYLDFIHVMTYDLHGSWEGYTGENSPLYKYPTETGSNAYLNVDYVMNYWKNNGAPAEKLIVGFPEYGHTFILRNPSDNGIGAPTSGDGPAGPYTRQAGFWAYYEICTFLRSGATEVWDASQEVPYAYKANEWLGYDNIKSFSVKAQWLKQNNFGGAMIWAIDLDDFTGSFCDQGKFPLTSTLNKALGISTEGCTAPDVPSEPVTTPPGSGSGGGSSGGSSGGSGFCADKADGLYPVADDRNAFWQCINGITYQQHCQAGLVFDTSCNCCNWP

SEQ ID NO: 12 - protein, 464 aa, Mus musculus
MVQSLAWAGVMTLLMVQWGSAAKLVCYLTNWSQYRTEAVRFFPRDVDPNLCTHVIFAFAGMDNHQLSTVEHNDELLYQELNSLKTKNPKLKTLLAVGGWTFGTQKFTDMVATASNRQTFVKSALSFLRTQGFDGLDLDWEFPGGRGSPTVDKERFTALIQDLAKAFQEEAQSSGKERLLLTAAVPSDRGLVDAGYEVDKIAQSLDFINLMAYDFHSSLEKTTGHNSPLYKRQGESGAAAEQNVDAAVTLWLQKGTPASKLILGMPTYGRSFTLASSSDNGVGAPATGPGAPGPYTKDKGVLAYYEACSWKERHRIEDQKVPYAFQDNQWVSFDDVESFKAKAAYLKQKGLGGAMVWVLDLDDFKGSFCNQGPYPLIRTLRQELNLPSETPRSPEQIIPEPRPSSMPEQGPSPGLDNFCQGKADGVYPNPGDESTYYNCGGGRLFQQSCPPGLVFRASCKCCTWS

SEQ ID NO: 13 - protein, 390 aa, Homo sapiens
MGATTMDQKSLWAGVVVLLLLQGGSAYKLVCYFTNWSQDRQEPGKFTPENIDPFLCSHLIYSFASIENNKVIIKDKSEVMLYQTINSLKTKNPKLKILLSIGGYLFGSKGFHPMVDSSTSRLEFINSIILFLRNHNFDGLDVSWIYPDQKENTHFTVLIHELAEAFQKDFTKSTKERLLLTAGVSAGRQMIDNSYQVEKLAKDLDFINLLSFDFHGSWEKPLITGHNSPLSKGWQDRGPSSYYNVEYAVGYWIHKGMPSEKVVMGIPTYGHSFTLASAETTVGAPASGPGAAGPITESSGFLAYYEICQFLKGAKITRLQDQQVPYAVKGNQWVGYDDVKSMETKVQFLKNLNLGGAMIWSIDMDDFTGKSCNQGPYPLVQAVKRSLGSL

SEQ ID NO: 14 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRTLGQQVPYATKGNQWVGYDDQESVKSKVQYLKDRQLAGAMVWALDLDDFQGSFCGQDLRFPLTNAIKDALAAT

SEQ ID NO: 15 - protein, 473 aa, Homo sapiens; identical to SEQ ID NO: 11

SEQ ID NO: 16 - protein, 466 aa, Homo sapiens
MVRSVAWAGFMVLLMIPWGSAAKLVCYFTNWAQYRQGEARFLPKDLDPSLCTHLIYAFAGMTNHQLSTTEWNDETLYQEFNGLKKMNPKLKTLLAIGGWNFGTQKFTDMVATANNRQTFVNSAIRFLRKYSFDGLDLDWEYPGSQGSPAVDKERFTTLVQDLANAFQQEAQTSGKERLLLSAAVPAGQTYVDAGYEVDKIAQNLDFVNLMAYDFHGSWEKVTGHNSPLYKRQEESGAAASLNVDAAVQQWLQKGTPASKLILGMPTYGRSFTLASSSDTRVGAPATGSGTPGPFTKEGGMLAYYEVCSWKGATKQRIQDQKVPYIFRDNQWVGFDDVESFKTKVSYLKQKGLGGAMVWALDLDDFAGFSCNQGRYPLIQTLRQELSLPYLPSGTPELEVPKPGQPSEPEHGPSPGQDTFCQGKADGLYPNPRERSSFYSCAAGRLFQQSCPTGLVFSNSCKCCTWN